SEQUENCE LISTING (15 sequences)

SEQ ID NO: 1 - protein, 972 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 2 - DNA, 2920 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatattaaaatcatccagaataaagatgccaaaatcgcagtcagtattttttatcccaatacattaaacggcttagtgaaaaaactaaacaatattattgaatataataaaaatatattcgttattgttctacatgttgataagaatcatcttacaccagatatcaaaaaagaaatactagccttctatcataaacatcaagtgaatattttactaaataatgatatctcatattacacgagtaatagattaataaaaactgaggcgcatttaagtaatattaataaattaagtcagttaaatctaaattgtgaatacatcatttttgataatcatgacagcctattcgttaaaaatgacagctatgcttatatgaaaaaatatgatgtcggcatgaatttctcagcattaacacatgattggatcgagaaaatcaatgcgcatccaccatttaaaaagctcattaaaacttattttaatgacaatgacttaaaaagtatgaatgtgaaaggggcatcacaaggtatgtttatgacgtatgcgctagcgcatgagcttctgacgattattaaagaagtcatcacatcttgccagtcaattgatagtgtgccagaatataacactgaggatatttggttccaatttgcacttttaatcttagaaaagaaaaccggccatgtatttaataaaacatcgaccctgacttatatgccttgggaacgaaaattacaatggacaaatgaacaaattgaaagtgcaaaaagaggagaaaatatacctgttaacaagttcattattaatagtataactctataaa

SEQ ID NO: 3 - protein, 965 aa, Pasteurella multocida
MNTLSQAIKAYNSNDYELALKLFEKSAETYGRKIVEFQIIKCKEKLSTNSYVSEDKKNSVCDSSLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNENIENAVDYDMFLKLSEVGKFKHLNKICYNRVLHGDNTSIKKLGIQKKNHFVVVNQSLNRQGINYYNYDKFDDLDESRKYIFNKTAEYQEEMDILKDLKLIQNKDAKIAVSIFYPNTLNGLVKKLNNIIEYNKNIFVIILHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLRSMNVKGASQGMFMKYALPHELLTIIKEVITSCQSIDSVPEYNTEDIWFQFALLILEKKTGHVFNKTSTLTYMPWERKLQWTNEQIQSAKKGENIPVNKFIINSITL

SEQ ID NO: 4 - DNA, 2979 bp, Pasteurella multocida
ttataaactgattaaagaaggtaaacgattcaagcaaggttaatttttaaaggaaagaaaatgaatacattatcacaagcaataaaagcatataacagcaatgactatgaattagcactcaaattatttgagaagtctgctgaaacctacgggcgaaaaatcgttgaattccaaattatcaaatgtaaagaaaaactctcgaccaattcttatgtaagtgaagataaaaaaaacagtgtttgcgatagctcattagatatcgcaacacagctcttactttccaacgtaaaaaaattaactctatccgaatcagaaaaaaacagtttaaaaaataaatggaaatctatcactgggaaaaaatcggagaacgcagaaatcagaaaggtggaactagtacccaaagattttcctaaagatcttgttcttgctccattgccagatcatgttaatgattttacatggtacaaaaatcgaaaaaaaagcttaggtataaagcctgtaaataagaatatcggtctttctattattattcctacatttaatcgtagccgtattttagatataacgttagcctgtttggtcaatcagaaaacaaactacccatttgaagtcgttgttgcagatgatggtagtaaggaaaacttacttaccattgtgcaaaaatacgaacaaaaacttgacataaagtatgtaagacaaaaagattatggatatcaattgtgtgcagtcagaaacttaggtttacgtacagcaaagtatgattttgtctcgattctagactgcgatatggcaccacaacaattatgggttcattcttatcttacagaactattagaagacaatgatattgttttaattggacctagaaaatatgtggatactcataatattaccgcagaacaattccttaacgatccatatttaatagaatcactacctgaaaccgctacaaataacaatccttcgattacatcaaaaggaaatatatcgttggattggagattagaacatttcaaaaaaaccgataatctacgtctatgtgattctccgtttcgttattttagttgcggtaatgttgcattttctaaagaatggctaaataaagtaggttggttcgatgaagaatttaatcattgggggggcgaagatgtagaatttggttacagattatttgccaaaggctgttttttcagagtaattgacggcggaatggcataccatcaagaaccacctggtaaagaaaatgaaacagaccgcgaagctggtaaaagtattacgcttaaaattgtgaaagaaaaggtaccttacatctatagaaagcttttaccaatagaagattcacatattcatagaatacctttagtttctatttatatccccgcttataactgtgcaaattatattcaaagatgtgtagatagtgctcttaatcaaactgttgtcgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccattttagaatgtttacgattagagcttggcatttaacggatggatttaacgaaaatattgaaaacgccgtggattatgacatgttccttaaactcagtgaagttggaaaatttaaacatcttaataaaatctgctataaccgcgtattacatggtgataacacatccattaagaaactcggcattcaaaagaaaaaccattttgttgtagtcaatcagtcattaaatagacaaggcatcaattattataattatgacaaatttgatgatttagatgaaagtagaaagtatatcttcaataaaaccgctgaatatcaagaagaaatggatattttaaaagatcttaaactcattcaaaataaagatgccaaaatcgcagtcagtattttctatcccaatacattaaacggcttagtgaaaaaactaaacaatattattgaatataataaaaatatattcgttattattctacatgttgataagaatcatcttacaccagacatcaaaaaagaaatattggctttctatcataagcaccaagtgaatattttactaaataatgacatctcatattacacgagtaatagactaataaaaactgaggcacatttaagtaatattaataaattaagtcagttaaatctaaattgtgaatacatcatttttgataatcatgacagcctattcgttaaaaatgacagctatgcttatatgaaaaaatatgatgtcggcatgaatttctcagcattaacacatgattggatcgagaaaatcaatgcgcatccaccatttaaaaagctgattaaaacctattttaatgacaatgacttaagaagtatgaatgtgaaaggggcatcacaaggtatgtttatgaagtatgcgctaccgcatgagcttctgacgattattaaagaagtcatcacatcctgccaatcaattgatagtgtgccagaatataacactgaggatatttggttccaatttgcacttttaatcttagaaaagaaaaccggccatgtatttaataaaacatcgaccctgacttatatgccttgggaacgaaaattacaatggacaaatgaacaaattcaaagtgcaaaaaaaggcgaaaatatccccgttaacaagttcattattaatagtataacgctataaaacatttgcattttattaaaa

SEQ ID NO: 5 - protein, 617 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 6 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 7 - protein, 651 aa, Pasteurella multocida
MKRKKEMTQKQMTKNPPQHEKENELNTFQNKIDSLKTTLNKDIISQQTLLAKQDSKHPLSASLENENKLLLKQLQLVLQEFEKIYTYNQALEAKLEKDKQTTSITDLYNEVAKSDLGLVKETNSVNPLVSIIMTSHNTAQFIEASINSLLLQTYKNIEIIIVDDDSSDNTFEIASRIANTTSKVRVFRLNSNLGTYFAKNTGILKSKGDIIFFQDSDDVCHHERIERCVNILLANKETIAVRCAYSRLAPETQHIIKVNNMDYRLGFITLGMHRKVFQEIGFFNCTTKGSDDEFFHRIAKYYGKEKIKNLLLPLYYNTMRENSLFTDMVEWIDNHNIIQKMSDTRQHYATLFQAMHNETASHDFKNLFQFPRIYDALPVPQEMSKLSNPKIPVYINICSIPSRIAQLRRIIGILKNQCDHFHIYLDGYVEIPDFIKNLGNKATVVHCKDKDNSIRDNGKFILLEELIEKNQDGYYITCDDDIIYPSDYINTMIKKLNEYDDKAVIGLHGILFPSRMTKYFSADRLVYSFYKPLEKDKAVNVLGTGTVSFRVSLFNQFSLSDFTHSGMADIYFSLLCKKNNILQICISRPANWLTEDNRDSETLYHQYRDNDEQQTQLIMENGPWGYSSIYPLVKNHPKFTDLIPCLPFYFL

SEQ ID NO: 8 - DNA, 780 bp, Pasteurella multocida
aacaggggataaggtcagtaaatttaggatgatttttgactaatggataaatacttgaatatccccatggaccgttttccatgatcagctgagtttgttgctcatcattgtctcgatattgatgatagagtgtttcgctgtctctattatcttccgttagccagtttgctggtcttgaaatacaaatctgaagaatattatttttcttacacaagagagagaaatagatatcagccatgcctgaatgggtaaagtcagaaagagaaaattgattaaagagactgactctaaagctaacagttcctgtacctaatacattgaccgctttgtctttttccagaggtttatagaagctatataccagtctatccgccgaaaaatatttggtcattctacttggaaagagaatgccgtgtaaaccaataaccgctttatcatcgtattcattcagcttcttgatcatcgtattgatgtaatcgcttggatagataatgtcatcatcacaggttatataatatccatcttgatttttttcaatcaactcttccagtaaaatgaatttgccattatctctaatggagttatctttatctttgcaatgaacaacggttgctttattacctaaattttttatgaagtcagggatttctacatagccatcaagataaatatgaaaatgatcacattgattttttagtatgccgataatacgtcgtaattgcgctattcttgagggaatagaacaaatattgatataaacaggaatcttaggattggaca

SEQ ID NO: 9 - protein, 5 aa, Artificial Sequence
  Motif between PmHAS and Group A HAS
XDGSX

SEQ ID NO: 10 - protein, 5 aa, Artificial Sequence
  Motif between PmHAS and Group A HAS
DSDXY

SEQ ID NO: 11 - protein, 703 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDI

SEQ ID NO: 12 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 13 - protein, 541 aa, Pasteurella multocida
ISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 14 - protein, 695 aa, Pasteurella multocida
MNTLSQAIKAYNSNDYELALKLFEKSAETYGRKIVEFQIIKCKEKLSTNSYVSEDKKNSVCDSSLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNENIENAVDYDMFLKLSEVGKFKHLNKICYNRVLHGDNTSIKKLGIQKKNHFVVVNQSLNRQGINYYNYDKFDDLDESRKYIFNKTAEYQEEMDILKD

SEQ ID NO: 15 - protein, 651 aa, Pasteurella multocida
KLSTNSYVSEDKKNSVCDSSLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNENIENAVDYDMFLKLSEVGKFKHLNKICYNRVLHGDNTSIKKLGIQKKNHFVVVNQSLNRQGINYYNYDKFDDLDESRKYIFNKTAEYQEEMDILKD

(19) United States
(12) Patent Application Publication
(10) Pub. No.: US 2006/0105431 A1
(43) Pub. Date: May 18, 2006

(54) Polymer grafting by polysaccharide synthases using artificial sugar acceptors

(76) Inventor: Paul L. DeAngelis, Edmond, OK (US)

Correspondence Address: DUNLAP, CODDING & ROGERS P.C., PO BOX 16370, OKLAHOMA CITY, OK, 73113, US

(21) Appl. No.: 11/253,453

(22) Filed: Oct. 19, 2005

Related U.S. Application Data
(63) Continuation-in-part of application No. 11/178,560, filed on Jul. 11, 2005.
Application No. 11/178,560 is a continuation of application No. 10/184,485, filed on Jun. 27, 2002, now abandoned.
Application No. 10/184,485 is a continuation of application No. 09/437,277, filed on Nov. 10, 1999, now Pat. No. 6,444,447.
Application No. 10/184,485 is a continuation-in-part of application No. 09/283,402, filed on Apr. 1, 1999, now abandoned.
(63) Continuation-in-part of application No. 10/814,752, filed on Mar. 31, 2004.
(63) Continuation-in-part of application No. 10/142,143, filed on May 8, 2002.
(63) Continuation-in-part of application No. 11/042,530, filed on Jan. 24, 2005.
Application No. 11/042,530 is a continuation of application No. 09/842,484, filed on Apr. 25, 2001, now abandoned.
(60) Provisional application No. 60/620,162, filed on Oct. 19, 2004.
(60) Provisional application No. 60/107,929, filed on Nov. 11, 1998.
(60) Provisional application No. 60/458,939, filed on Mar. 31, 2003.
(60) Provisional application No. 60/289,554, filed on May 8, 2001.
(60) Provisional application No. 60/296,386, filed on Jun. 6, 2001.
(60) Provisional application No. 60/303,691, filed on Jul. 6, 2001.
(60) Provisional application No. 60/313,258, filed on Aug. 17, 2001.
(60) Provisional application No. 60/199,538, filed on Apr. 25, 2000.

(51) Int. Cl. C12P 19/28; C12N 9/24

(52) U.S. Cl. 435/85; 435/200

(57) ABSTRACT

The present invention relates to methodology for polymer grafting by a polysaccharide synthase and, more particularly, polymer grafting using the glycosaminoglycan synthases from Pasteurella multocida. The methodology of the present invention includes providing an enzymatically active glycosaminoglycan synthase enzyme from Pasteurella multocida, providing a synthetic, artificial acceptor for the glycosaminoglycan synthase enzyme; combining the synthetic, artificial acceptor with the glycosaminoglycan synthase enzyme within a reaction medium, wherein the reaction medium contains at least one sugar precursor selected from the group consisting of UDP-GlcA, UDP-GlcNAc, UDP-GalNAc, and reacting the glycosaminoglycan synthase enzyme with the synthetic, artificial acceptor to produce an oligosaccharide or polysaccharide polymer. The glycosaminoglycan synthase enzyme may be hyaluronan synthase, chondroitin synthase, or heparosan synthase from P. multocida, and the oligosaccharide or polysaccharide polymer may be hyaluronic acid (hyaluronan), chondroitin, heparosan, or combinations thereof.

### CROSS-REFERENCE TO RELATED APPLICATIONS

This application claims priority under 35 U.S.C. 119(e) of provisional application U.S. Ser. No. 60/620,162, filed Oct. 19, 2004. This application is also a continuation-in-part of U.S. Ser. No. 11/178,560, filed Jul. 11, 2005; which is a continuation of U.S. Ser. No. 10/184,485, filed Jun. 27, 2002, now abandoned; which is a continuation of U.S. Ser. No. 09/437,277, filed Nov. 10, 1999, now U.S. Pat. No. 6,444,447, issued Sep. 3, 2002; which claims benefit under 35 U.S.C. 119(e) of provisional application U.S. Ser. No. 60/107,929, filed Nov. 11, 1998. Said U.S. Ser. No. 09/437,277 is also a continuation-in-part of U.S. Ser. No. 09/283,402, filed Apr. 1, 1999, now abandoned.

This application is also a continuation-in-part of U.S. Ser. No. 10/814,752, filed Mar. 31, 2004; which claims priority under 35 U.S.C. § 119(e) of U.S. Provisional Application Ser. No. 60/458,939, filed Mar. 31, 2003, and is also a continuation-in-part of U.S. Ser. No. 10/142,143, filed May 8, 2002; which claims priority under 35 U.S.C. § 119(e) of U.S. Provisional Application Ser. No. 60/289,554, filed May 8, 2001; Ser. No. 60/296,386, filed Jun. 6, 2001; Ser. No. 60/303,691, filed Jul. 6, 2001; and Ser. No. 60/313,258, filed Aug. 17, 2001.

This application is also a continuation-in-part of U.S. Ser. No. 11/042,530, filed Jan. 24, 2005; which is a continuation of U.S. Ser. No. 09/842,484, filed Apr. 25, 2001, now abandoned; which claims priority under 35 U.S.C. 119(e) to U.S. provisional application Ser. No. 60/199,538, filed Apr. 25, 2000.

The entire contents of each of the above-referenced patents and applications are hereby expressly incorporated herein by reference in their entirety.

### STATEMENT REGARDING FEDERALLY SPONSORED RESEARCH OR DEVELOPMENT

At least a portion of the invention was developed under funding from the National Science Foundation under Grant No. MCB-9876193. As such, the Government may own certain rights in and to this application.

### BACKGROUND

1. Field of the Invention

The present invention relates to methodology for polymer grafting by a polysaccharide synthase and, more particularly, polymer grafting using the glycosaminoglycan (GAG) synthases from Pasteurella multocida. The present invention also relates to coatings for biomaterials wherein the coatings provide protective properties to the biomaterial and/or act as a bioadhesive. Such coatings could be applied to electrical devices, sensors, catheters and any device which may be contemplated for use within a mammal. The present invention further relates to drug delivery agents which are biocompatible and may comprise combinations of a GAG biomaterial or a bioadhesive and a medicament or a medicament-containing liposome. The biomaterial and/or bioadhesive may be a hyaluronic acid polymer produced by a hyaluronate synthase from Pasteurella multocida, a chondroitin polymer produced by a chondroitin synthase from Pasteurella multocida, or a heparosan polymer produced by a heparosan synthase from Pasteurella multocida. The present invention also relates to the creation of chimeric molecules containing GAG chains attached to various compounds, and especially artificial carbohydrate mimics. These artificial compounds may be in turn be attached to other soluble molecules or attached to surfaces.

2. Description of the Related Art

Polysaccharides are large carbohydrate molecules composed from about 25 sugar units to thousands of sugar units. Animals, plants, fungi and bacteria produce an enormous variety of polysaccharide structures which are involved in numerous important biological functions such as structural elements, energy storage, and cellular interaction mediation. Often, the polysaccharide's biological function is due to the interaction of the polysaccharide with proteins such as receptors and growth factors. The glycosaminoglycan class of polysaccharides, which includes heparin, chondroitin, and hyaluronic acid, play major roles in determining cellular behavior (e.g., migration, adhesion) as well as the rate of cell proliferation in mammals. These polysaccharides are, therefore, essential for correct formation and maintenance of organs of the human body.

Several species of pathogenic bacteria and fungi also take advantage of the polysaccharide's role in cellular communication. These pathogenic microbes form polysaccharide surface coatings or capsules that are identical or chemically similar to host molecules. For instance, Group A & C Streptococcus and Type A Pasteurella multocida produce authentic hyaluronic acid capsules and pathogenic Escherchia coli and Type F and D Pasteurella multocida are known to make capsules composed of polymers very similar to chondroitin and heparin. The pathogenic microbes form the polysaccharide surface coatings or capsules because such a coating is nonimmunogenic and protects the bacteria from host defenses thereby providing the equivalent of molecular camouflage.

Enzymes alternatively called synthases, synthetases, or transferases, catalyze the polymerization of polysaccharides found in living organisms. Many of the known enzymes also polymerize activated sugar nucleotides. The most prevalent sugar donors contain UDP but ADP, GDP, and CMP are also used depending on (1) the particular sugar to be transferred and (2) the organism. Many types of polysaccharides are found at, or outside of, the cell surface. Accordingly, most of the synthase activity is typically associated with either the plasma membrane on the cell periphery or the Golgi apparatus membranes that are involved in secretion. In general, these membrane-bound synthase proteins are difficult to manipulate by typical procedures and only a few enzymes have been identified after biochemical purification.

All of the known HA, chondroitin and heparan sulfate/heparin glycosyltransferase enzymes that synthesize the alternating sugar repeat backbones in microbes and in vertebrates utilize UDP-sugar precursors and metal cofactors (e.g., magnesium and/or manganese ion) near neutral pH according to the overall reaction:

n UDP-GlcUA+n UDP-HexNAc→2n UDP+[GlcUA-HexNAc]ₙ

where HexNAc=GlcNAc or GalNAc. Depending on the specific GAG and the particular organism or tissue examined, the degree of polymerization, n, ranges from ˜25 to ˜10,000. The bacterial GAG glycosyltransferase polypeptides are associated with the cell membranes; this localization makes sense with respect to synthesis of polysaccharide molecules destined for the cell surface.

Various names for the GAG glycosyltransferases have been used in the literature over the last four decades. The dual-action enzymes required for the production of the HA chain have been called synthases (or in early reports, synthetases). The enzymes that elongate the repeating chondroitin or the repeating heparan sulfate/heparin backbone have been called various names including copolymerases, cotransferases, polymerases, synthases, or the individual component activities were directly termed (e.g., GlcUA-transferase, GlcNAc-transferase or GalNAc-transferase).

The HA extracellular capsules of Gram-positive Group A Streptococcus (Kendall et al., 1937) and Gram-negative Type A Pasteurella multocida (Carter and Annau, 1953) were shown to be identical to HA of vertebrates. As the vertebrate HA synthases were (and remain) relatively difficult to study biochemically, more initial progress was made on the “simpler,” higher specific activity membrane preparations of streptococcal enzymes (Stoolmiller and Dorfman, 1969; Sugahara et al., 1979).

Transposon insertional mutagenesis was utilized to tag and to identify the genes for the microbial HA synthases [HASs] of both Group A Streptococcus (S. pyogenes spHAS or HasA; DeAngelis et al., 1993; Dougherty and van de Rijn, 1994) and P. multocida Type A (pmHAS; DeAngelis et al., 1998). Degenerate PCR based on the Group A Streptococcus HAS sequence was used to obtain the homologous enzyme sequence from a Group C organism (S. equisimilis seHAS; Kumari and Weigel, 1997). In all the known cases (including the vertebrate and viral enzymes; reviewed in Weigel et al., 1997; DeAngelis, 1999a), the HA polysaccharide is polymerized by a single polypeptide, the HA synthase [HAS].

The microbial HASs contain two distinct glycosyltransferase activities as demonstrated by expression in foreign hosts (e.g., Escherichia coli) and various biochemical analyses (DeAngelis et al., 1993, 1998; DeAngelis and Weigel, 1994; Kumari and Weigel, 1997). Recombinant preparations of the microbial HA synthases rapidly form HA chains with elongation rates of ˜10-150 sugars/second in vitro.

The streptococcal enzymes and the Pasteurella enzyme produce the same polymer product from identical precursors, but these synthases possess quite distinct sequences and enzymological characteristics. The streptococcal HASs are integral membrane proteins with several transmembrane or membrane-associated regions (DeAngelis et al., 1993; Heldermon et al., 2001). Vertebrate HASs have similar sequence motifs and predicted structure to the streptococcal enzymes (reviewed in Weigel et al., 1997). On the other hand, the Pasteurella enzyme appears to contain a carboxyl-terminal region that allows docking with a membrane-bound partner because deletion of the region results in the expression of a functional soluble, cytoplasmic form of the enzyme (Jing and DeAngelis, 2000). As discussed later, recombinant pmHAS can elongate exogenously supplied HA-oligosaccharide acceptors, but the streptococcal and vertebrate enzymes have not been shown to perform similar reactions (Stoolmiller and Dorfman, 1969; DeAngelis, 1999b). In summary, two classes of HA synthase enzyme have been discovered thus far; Class I includes the streptococcal, vertebrate, and viral HASs, while the only Class II member is the enzyme from Pasteurella (DeAngelis, 1999a).

The chondroitin chain is chemically identical to HA except that GalNAc is substituted for GlcNAc. Certain distinct isolates of Pasteurella multocida, now called Type F, were speculated to produce a chondroitin-like polymer based on the sensitivity of the bacterial capsule to chondroitin ABC lyase (Rimler, 1994). The capsular polysaccharide contains GalNAc and a uronic acid (DeAngelis and Padgett-McCue, 2000) and is unsulfated chondroitin as assessed by structural analyses (DeAngelis, Gunay, Toida, Mao, and Linhardt; unpublished). Experiments utilizing pmHAS DNA probes and PCR primers indicated that a novel homologous synthase existed. An open reading frame, called pmCS, with ˜90% identity at the gene and protein level to pmHAS was shown to have chondroitin synthase activity in vitro (DeAngelis and Padgett-McCue, 2000). Recombinant pmCS polymerizes long chains (˜1000 sugars) composed of GalNAc and GlcUA that are sensitive to chondroitin ABC lyase but not HA lyase. The pmCS enzyme, like pmHAS, is a selective glycosyltransferase; only the authentic precursors, UDP-GalNAc and UDP-GlcUA, serve as donors in vitro.

An analogous E. coli enzyme, KfoC, with ˜70% identity to pmCS was discovered subsequently (Ninomiya et al., 2002), but in K4 the chondroitin polymer is fructosylated at C3 of the GlcUA groups. The vertebrate chondroitin synthase is not very similar at the DNA or protein sequence level to pmCS (Kitagawa et al., 2001).

Heparan sulfate/heparin and related polymers contain alternating α- and β-glycosidic linkages, and thus are quite distinct from the entirely β-linked HA and chondroitin polymers. The UDP-sugar precursors are β-linked; therefore, heparin biosynthesis exhibits two types of reaction pathways: a retaining mechanism to produce the α-linkage and an inverting mechanism that results in a β-glycosidic-linkage.

E. coli K5 produces a capsule composed of an unsulfated, unepimerized N-acetyl-heparosan (heparosan or desulfatoheparin) (Vann et al., 1981). The E. coli K5 capsular locus contains open reading frames KfiA-D (also called the Kfa locus in some reports; Petit et al., 1995). Biochemical analyses of the glycosyltransferase activities in membrane preparations or in lysates from both the native K5 and recombinant bacteria have been reported (Finke et al., 1991; Griffiths et al., 1998). However, it was difficult to ascertain that two distinct enzymes were actually required for the synthesis of the repeating GAG chain in part due to the lack of continued polymerization by recombinant enzymes in vitro; only the addition of single sugars to oligosaccharide acceptors was observed. At first, KfiC was stated to be a dual-action glycosyltransferase responsible for the alternating addition of both GlcUA and GlcNAc to the heparosan chain (Griffiths et al., 1998). This report also concluded that the enzyme's GlcUA-transferase activity was inactivated by the removal of a segment of the carboxyl terminus, but the GlcNAc-transferase activity remained intact. However, a later report by the same group reported that another protein, KfiA, encoded by the same operon was actually the α-GlcNAc-transferase required for heparosan polymerization (Hodson et al., 2000). Therefore, at least these two enzymes, KfiA and KfiC, work in concert to form the disaccharide repeat. Another deduced protein in the operon, KfiB, was suggested to stabilize the enzymatic complex during elongation in vivo, but not participate directly in catalysis.

The Type D Pasteurella multocida capsular polysaccharide is also N-acetylheparosan as measured by compositional and structural analyses (DeAngelis, Gunay, Toida, Mao, and Linhardt; unpublished). In this microbe, however, the polymer is synthesized by a dual-action glycosyltransferase, the heparosan synthase or pmHS1 (DeAngelis and White, 2002). Another similar (−73% identical) enzyme, pmHS2, was found in Types A, D, and F P. multocida (DeAngelis and White, 2004). The two recombinant E. coli-derived enzymes, pmHS1 or pmHS2, polymerize both GlcNAc and GlcUA to form the heparosan chain in vitro.

One region of the pmHS protein is similar to E. coli K5 KfiA while another region of pmHS is similar to KfiC suggesting that a two-domain structure exists in the Pasteurella enzyme. The sequence of pmHS, however, is very different from other Pasteurella GAG synthases, pmHAS and pmCS. The overall organization of the capsule loci of Type A, D, and F P. multocida, on the other hand, are quite similar based on recent sequence comparisons (Townsend et al., 2001). Most notably, highly homologous UDP-glucose dehydrogenase genes (92-98% identical) follow the synthase genes in all three capsular types. The exostosin proteins, EXT1 and 2, the vertebrate enzymes responsible for biosynthesis of the heparan sulfate/heparin backbone, are not similar to the bacterial heparosan glycosyltransferases at the sequence level (reviewed in Duncan et al., 2001).

TABLE I
Structures of the Glycosaminoglycan Repeating Sugar Backbones.

| Polymer | Disaccharide Repeat |
|---|---|
| Hyaluronan, HA | β3GlcNAcβ4GlcUA |
| Chondroitin | β3GalNAcβ4GlcUA |
| Heparosan | α4GlcNAcβ4GlcUA |

TABLE II
Microbes, GAGs, and Glycosyltransferases.

| | Hyal- | Chon- | Hepa- | Enzyme |
|---|---|---|---|---|
| Bacteria | uronan | droitin | rosan | [Size^{a}]/GenBank # |
| Streptococcus | | | | |
| Group A | X | | | spHAS [419]/L20853 |
| Group C | X | | | seHAS [418]/AF023876 |
| Escherichia coli | | | | |
| K4 | | X^{b} | | KfoC [686]/AB079602 |
| K5 | | | X | KfiA [238] + KfiC [520] |
| | | | | complex/X77617 |
| Pasteurella | | | | |
| multocida | | | | |
| Type A | X | | | pmHAS [972]/AF036004 |
| Type D | | | X | pmHS1 [617]/AF25591 |
| Types A, D, F | | | X | pmHS2 [651]/AY292199 |
| Type F | | X | | pmCS [965]/AF195517 |

^{a}number of amino acid residues in the deduced open reading frame.
^{b}fructosylated polymer.

A wide variety of polysaccharides are commercially harvested from many sources, such as xanthan from bacteria, carrageenans from seaweed, and gums from trees. This substantial industry supplies thousands of tons of these raw materials for a multitude of consumer products ranging from ice cream desserts to skin cream cosmetics. Vertebrate tissues and pathogenic bacteria are the sources of more exotic polysaccharides utilized in the medical field as surgical aids, vaccines, and anticoagulants. For example, two glycosaminoglycan polysaccharides, heparin from pig intestinal mucosa and hyaluronic acid from rooster combs, are employed in several applications including clot prevention and eye surgery, respectively. Polysaccharides extracted from bacterial capsules (e.g., various Streptococcus pneumoniae strains) are utilized to vaccinate both children and adults against disease with varying levels of success. However, for the most part, one must use the existing structures found in the raw materials as obtained from nature. In many of the older industrial processes, chemical modification (e.g., hydrolysis, sulfation, deacetylation) is used to alter the structure and properties of the native polysaccharide. However, the synthetic control and the reproducibility of large-scale reactions are not always successful.

Some of the current methods for designing and constructing carbohydrate polymers in vitro utilize: (i) difficult, multistep sugar chemistry, or (ii) reactions driven by transferase enzymes involved in biosynthesis, or (iii) reactions harnessing carbohydrate degrading enzymes catalyzing transglycosylation. The latter two methods are restricted by the specificity and the properties of the available naturally occurring enzymes. Many of these enzymes are neither particularly abundant nor stable but are almost always expensive. Overall, the procedures currently employed yield polymers containing between 2 and about 12 sugars. Unfortunately, many of the physical and biological properties of polysaccharides do not become apparent until the polymer contains 25, 100, or even thousands of monomers.

To facilitate the development of biotechnological medical improvements, the present invention provides a method to apply a surface coating of HA that will shield the artificial components or compounds from the detrimental responses of the body as well as encourage engrafting of a foreign medical device within living tissue. Such a coating of HA will bridge the gap between man-made substances and living flesh (i.e., improve biocompatibility). The HA can also be used as a biomaterial such as a biodhesive or a bioadhesive containing a medicament delivery system, such as a liposome, and which is non-immunogenic. As GAGs are recognized by certain cells, this biomaterial can also be used to target an attached medicament. The present invention also encompasses the methodology of polysaccharide polymer grafting, i.e., HA or chondroitin or heparosan, using either a hyaluronate synthase (PmHAS) or a chondroitin synthase (PmCS) or a heparosan synthase (PmHS1 or PmHS2) from P. multocida with the use of artificial acceptors. Modified versions of the PmHAS, PmCS or PmHS enzymes (genetic or chemical) can also be utilized to graft on polysaccharides of various size and composition.

### SUMMARY OF THE INVENTION

A unique HA synthase, PmHAS, from the fowl cholera pathogen, Type A P. multocida has been identified and cloned and is disclosed and claimed in co-pending U.S. Ser. No. 09/283,402, filed Apr. 1, 1999, and entitled “DNA Encoding Hyaluronan Synthase From Pasteurella multocida and Methods,” the contents of which are hereby expressly incorporated herein. The amino acid and nucleic acid sequences of PmHAS are shown in SEQ ID NOS:1 and 2, respectively. Expression of this single 972-residue protein allows Escherichia coli host cells to produce HA capsules in vivo; normally E. coli does not make HA. Extracts of recombinant E. coli, when supplied with the appropriate UDP-sugars, make HA in vitro. Thus, the PmHAS is an authentic HA synthase.

A chondroitin synthase has also been identified and molecularly cloned from P. multocida, and named pmCS (P. multocida Chondroitin Synthase), as disclosed and claimed in U.S. Ser. No. 09/842,484, filed Apr. 25, 2001, the contents of which are hereby expressly incorporated herein. The amino acid and nucleic acid sequences of PmCS are shown in SEQ ID NOS:3 and 4, respectively. This is the first chondroitin synthase to be identified and molecularly cloned from any source. The recombinant E. coli-derived enzyme PmCS polymerizes both GalNAc and GlcUA to form the chondroitin polymer in vitro.

In addition, two heparosan synthase have also been identified and molecularly cloned from P. multocida, as disclosed and claimed in U.S. Ser. No. 10/142,143, filed May 8, 2002, the contents of which are hereby expressly incorporated herein. PmHS1 was identified in Type D P. multocida, and this is the first heparosan synthase to be identified and molecularly cloned from any source. The amino acid and nucleic acid sequences of PmHS1 are shown in SEQ ID NOS:5 and 6, respectively. PmHS2 was subsequently identified and is found in Types A, D and F P. multocida. The amino acid and nucleic acid sequences of PmHS2 are shown in SEQ ID NOS:7 8nd 2, respectively. The two recombinant E. coli-derived enzymes, pmHS1 or PmHS2, polymerize both GlcNAc and GlcUA to form the heparosan chain in vitro.

It has also been determined that the P. multocida GAG synthases add sugars to the nonreducing end of a growing polymer chain. The correct monosaccharides are added sequentially in a stepwise fashion to the nascent chain or a suitable exogenous HA oligosaccharide. The PmHAS sequence, however, is significantly different from the other known HA synthases. There appears to be only two short potential sequence motifs ([D/N]DGS[S/T] (SEQ ID NO:9); DSD[D/T]Y (SEQ ID NO:10) in common between PmHAS and the Group A HAS—HasA. Instead, a portion of the central region of the new enzyme is more homologous to the amino termini of other bacterial glycosyltransferases that produce different capsular polysaccharides or lipopolysaccharides.

When the PmHAS is given long elongation reaction times, HA polymers of at least 400 sugars long are formed. Unlike any other known HAS enzyme, PmHAS also has the ability to extend exogenously supplied short HA oligosaccharides into long HA polymers in vitro. If enzyme is supplied with these short HA oligosaccharides, total HA biosynthesis is increased up to 50-fold over reactions without the exogenous oligosaccharide. The nature of the polymer retention mechanism of the PmHAS polypeptide might be the causative factor for this activity: i.e., a HA-binding site may exist that holds onto the HA chain during polymerization. Small HA oligosaccharides also, are capable of occupying this site of the recombinant enzyme and thereafter be extended into longer polysaccharide chains.

Most membrane proteins are relatively difficult to study due to their insolubility in aqueous solution, and the HASs are no exception. Only the enzyme from Group A and C Streptococcus bacteria has been detergent-solubilized and purified in an active state in small quantities. Once isolated in a relatively pure state, the streptococcal enzyme has very limited stability. A soluble recombinant form of the enzyme from P. multocida called PmHAS-D which comprises residues 1-703 of the 972 residues of the native PmHAS enzyme, the amino acid sequence of PmHAS-D is shown in SEQ ID NO:11 with the nucleotide sequence of PmHAS-D is shown in SEQ ID NO: 12. PmHAS-D can be mass-produced in E. coli and purified by chromatography. The PmHAS-D enzyme retains the ability of the parent enzyme to add on a long HA polymer onto short HA primers. Furthermore, the purified PmHAS-D enzyme is stable in an optimized buffer for days on ice and for hours at normal reaction temperatures. In an analogous fashion, PmHS1 or PmCS may be truncated (for example but not by way of limitation, PmHS1₇₇₋₆₁₇ (SEQ ID NO:13), PmCS₁₋₆₉₅ (SEQ ID NO:14) or PmCS₄₅₋₆₉₅ (SEQ ID NO:15)) and used in vitro. One formulation of the optimal buffer consists of 1M ethylene glycol, 0.1-0.2 M ammonium sulfate, 50 mM Tris, pH 7.2, and protease inhibitors which allows the stability and specificity at typical reaction conditions for sugar transfer. For the reaction UDP-sugars and manganese (10-20 mM) are added. PmHAS-D will also add on a HA polymer onto plastic beads with an immobilized short HA primer.

The present invention encompasses methods of producing a variety of unique biocompatible molecules and coatings based on oligosaccharides and polysaccharides. Polysaccharides, especially those of the glycosaminoglycan class, serve numerous roles in the body as structural elements and signaling molecules. The GAG oligosaccharides also have biological activities. By grafting or making hybrid molecules composed of more than one polymer backbone, it is possible to meld distinct physical and biological properties into a single molecule without resorting to unnatural chemical reactions or residues.

The present invention also incorporates the propensity of certain recombinant enzymes, when prepared in a virgin state, to utilize various acceptor molecules as the seed for further polymer growth: naturally occurring forms of the enzyme or existing living host organisms do not display this ability. Thus, the present invention results in (a) the production of hybrid polysaccharides and (b) the formation of polysaccharide coatings. Such hybrid polymers can serve as “molecular glue”—i.e., when two cell types or other biomaterials interact with each half of a hybrid molecule, then each of the two phases are bridged.

Such polysaccharide coatings are useful for integrating a foreign object within a surrounding tissue matrix. For example, a prosthetic device is more firmly attached to the body when the device is coated with a naturally adhesive polysaccharide. Additionally, the devices artificial components could be masked by the biocompatible coating to reduce immunoreactivity or inflammation. Another aspect of the present invention is the coating or grafting of HA or chondroitin or heparosan onto various drug delivery matrices or bioadhesives or suitable medicaments to improve and/or alter delivery, half-life, persistence, targeting and/or toxicity.

The present invention also demonstrates the identification of new artificial acceptors or primers for the GAG synthases of P. multocida that allow simpler, less expensive, animal-free processes to be utilized in the production of oligosaccharide or polysaccharide polymers. The small molecules of the present invention can substitute for the HA oligosaccharides described and claimed in the inventor's technology described in U.S. Pat. No. 6,444,447, which has previously been incorporated herein by reference. As described herein, not all artificial sugar mimics are practical, and thus the present invention is not obvious based on previous experiments by others with GAG synthases.

The present invention provides methods for producing a glycosaminoglycan polymer derivative. The methods include providing an enzymatically active glycosaminoglycan synthase enzyme from Pasteurella multocida; providing a synthetic, artificial acceptor for the glycosaminoglycan synthase enzyme; combining the synthetic, artificial acceptor with the glycosaminoglycan synthase enzyme within a reaction medium, wherein the reaction medium contains at least one sugar precursor selected from the group consisting of UDP-GlcA, UDP-GlcNAc, and UDP-GalNAc, or a monosaccharide mimic with the functional groups found in GlcA, GlcNAc and GalNAc; and reacting the glycosaminoglycan synthase enzyme with the synthetic, artificial acceptor to produce an oligosaccharide or polysaccharide polymer derivative.

The glycosaminoglycan synthase enzyme may be hyaluronan synthase, chondroitin synthase, or heparosan synthase, or combinations thereof. The oligosaccharide or polysaccharide polymer derivative may be a hyaluronic acid polymer derivative, a chondroitin polymer derivative, a heparosan polymer derivative, or combinations thereof.

The synthetic, artificial acceptor may comprise at least one monosaccharide attached to an organic hydrophobic molecule, such as but not limited to, fluorescein di-β-D-glucuronide (A-F-A), β-trifluoromethylumbelliferyl β-D-glucuronide (A-F₃MUM), and 4-methylumbelliferyl N-acetyl-β-D-glucosaminide (N-MUM). In a preferred embodiment, the synthetic, artificial acceptor may comprise two GlcA sugars attached to an aromatic nucleus. In a more preferred embodiment, the synthetic, artificial acceptor may be A-F-A (fluorescein di-βD-glucuronide).

### DESCRIPTION OF THE DRAWINGS

FIG. 1 is a graphical representation showing that an HA tetramer stimulates PmHAS polymerization.

FIG. 2 is a graphical plot showing that HA polymerization is effected by HA oligosaccharides.

FIG. 3 is a graphical plot showing HA tetramer elongation into larger polymers by PmHAS-D.

FIG. 4 is a graphical representation of a thin layer chromatography analysis of PmHAS extension of HA tetramer.

FIG. 5 are graphical plots of a time course of single sugar addition to native HA oligosaccharides. The reactions were carried out as described for single sugar addition and analyzed using descending paper chromatography. Panel A: Two independent reactions (open or solid symbols) were monitored over time. Under virtually identical conditions, the GlcA-transferase activity of PmHAS (triangles) is ˜20-fold more rapid than that of the GlcNAc-transferase activity (circles) in vitro. Panel B: magnified scale to depict linearity of GlcNAc-transferase activity.

FIG. 6 is a Michaelis-Menten analysis of methoxyphenol sugars as acceptors. Single sugar addition reactions were performed in duplicate using MP-oligosaccharides and then purified using SPE; averaged data minus the “no acceptor control” (<0.1 pmol/min) is shown. Panel A: GlcA-transferase activity assays with GlcNAc-terminated acceptors: NANAN-MP (open circles), NANA-MP (solid circles), NAN-MP (solid squares), and NA-MP (open triangles). Panel B: GlcNAc-transferase activity assays with GlcA-terminated acceptors: ANAN-MP (open and solid triangles), ANANAN-MP (solid squares), ANA-MP (open circles), and AN-MP (solid circles).

FIG. 7 is a gel analysis of polymer products using A-F-A acceptor. Three parallel chemoenzymatic HA synthesis reactions with different concentrations of A-F-A were separated on a 1.2% agarose gel with Stains-All detection. The material is authentic HA as shown by its sensitivity to HA lyase treatment (+L). The UDP-sugar/acceptor stoichiometry of the reaction controls the size of the HA polymer product. (S=HA standards: ranging from 27-1510 kDa from bottom to top).

FIG. 8 is a mass spectrogram demonstrating that the PmHAS enzyme converted AFA, an artificial acceptor, into a product which is AFA with a single GlcNAc sugar addition (AFA-N) from the UDP-GlcNAc precursor. A small amount of product with two GlcNAc groups, N-AFA-N, is also formed.

FIG. 9 illustrates the chemical structures of various candidate acceptor molecules.

### DETAILED DESCRIPTION OF THE INVENTION

Before explaining at least one embodiment of the invention in detail, it is to be understood that the invention is not limited in its application to the details of construction and the arrangements of the components set forth in the following description or illustrated in the drawings. The invention is capable of other embodiments or of being practiced or carried out in various ways. Also, it is to be understood that the phraseology and terminology employed herein is for purpose of description and should not be regarded as limiting.

The term “synthetic, artificial acceptor” as used herein will be understood to refer to a sugar-containing compound that does not contain the naturally occurring disaccharide repeat, and thus when extended by a GAG synthase produces a non-naturally occurring glycosaminoglycan derivative. Thus, the new acceptor serves as a GAG mimic; the complex naturally occurring sugar does not need to be synthesized or extracted for use as an acceptor. Examples of synthetic artificial acceptors that may be utilized in accordance with the present invention, along with the abbreviations utilized herein, include but are not limited to, fluorescein mono-β-D-glucuronide (A-F); fluorescein di-β-D-glucuronide (A-F-A); p-nitrophenyl-β-D-glucuronide (A-NP); 4-methylumbelliferyl β-D-glucuronide (A-MUM); β-trifluoromethylumbelliferyl β-D-glucuronide (A-F₃MUM); and 4-methylumbelliferyl N-acetyl-β-D-glucosaminide (N-MUM). Other artificial compounds utilized herein that are not necessarily practical artificial acceptors include fluorescein di-β-D-glucopyranoside (G-F-G); fluorescein di-β-D-N-acetylgalactosamine (GalNAc-F-GalNAc); 1-naphthyl β-D-glucuronide (A-NAP); 4-nitrophenyl-β-D-galacturonide (GalA-NP); 3-carboxyumbelliferyl P-D-glucuronide (A-CU); and 4-methylumbelliferyl SD-glucopyranoside (G-MUM).

The term “glycosaminoglycan derivative” as used herein will be understood to refer to an oligosaccharide or polysaccharide glycosaminoglycan polymer having a synthetic, artificial acceptor attached to one end thereof. The term “hyaluronic acid polymer derivative” as used herein will be understood to refer to an hyaluronic acid oligosaccharide or polysaccharide polymer having a synthetic, artificial acceptor attached to one end thereof. The term “chondroitin polymer derivative” as used herein will be understood to refer to a chondroitin oligosaccharide or polysaccharide polymer having a synthetic, artificial acceptor attached to one end thereof. The term “heparosan polymer derivative” as used herein will be understood to refer to a heparosan oligosaccharide or polysaccharide polymer having a synthetic, artificial acceptor attached to one end thereof. In one embodiment, the glycosaminoglycan derivative, hyaluronic acid polymer derivative, chondroitin synthase polymer derivative or heparosan polymer derivative may be a glycoside.

The term “glycoside” as used herein refers to a type of compound containing a saccharide (e.g., a monosaccharide or longer sugar chain, etc) attached to a non-carbohydrate molecule (e.g., a hydrophobic organic compound, etc.) via its reducing terminus (i.e., through a bond at the Carbon-1 position).

The term “organic hydrophobic molecule” as used herein will be understood to refer to a relatively non-polar compound that contains an abundance of C—H bonds.

The term “aromatic nucleus” as used herein will be understood to refer to an organic compound containing one or more benzene-like rings (i.e., alternating single and double C—C bonds).

In the present invention, a coding scheme is utilized herein to refer to HA-like oligosaccharides constructed in accordance with the present invention. For simplicity, the coding scheme employs “A” to designate the glucuronic acid or GlcA monosaccharide and “N” to designate the N-acetyl-glucosamine or GlcNAc monosaccharide. The coding scheme also employs “MP” to designate the simple aromatic compound, methoxyphenyl. For example, the compound AN-MP refers to GlcA-GlcNAc-MP as read from non-reducing end to reducing end. In another example, the compound NA-MP refers to GlcNAc-GlcA-MP as read from non-reducing end to reducing end.

The terms “UDP-GlcA and “UDP-GlcUA” are used interchangeably herein, as UDP-GlcA is a new version of the older abbreviation UDP-GlcUA. Both terms are used herein to designate glucuronic acid.

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a Hyaluronic Acid Synthase (“HAS”) coding sequence or Chondroitin Synthase (“CS”) coding sequence or a Heparosan Synthase (“HS”) coding sequence yet is isolated away from, or purified free from, unrelated genomic DNA, for example, total Pasteurella multocida. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified PmHAS-D or PmCS or PmHS1 or PmHS2 gene refers to a DNA segment including HAS or chondroitin synthase or heparosan synthase coding sequences isolated substantially away from other naturally occurring genes or protein encoding sequences. In this respect, the term “gene” is used for simplicity to refer to a functional protein, polypeptide or peptide encoding unit. As will be understood by those in the art, this functional term includes genomic sequences, cDNA sequences or combinations thereof. “Isolated substantially away from other coding sequences” means that the gene of interest, in this case PmHAS-D or PmCS or PmHS1 or PmHS2, forms the significant part of the coding region of the DNA segment, and that the DNA segment does not contain large portions of naturally-occurring coding DNA, such as large chromosomal fragments or other functional genes or DNA coding regions. Of course, this refers to the DNA segment as originally isolated, and does not exclude genes or coding regions later added to, or intentionally left in the segment by the hand of man.

Due to certain advantages associated with the use of prokaryotic sources, one will likely realize the most advantages upon isolation of the HAS or chondroitin synthase or heparosan synthase gene from the prokaryote P. multocida. One such advantage is that, typically, eukaryotic enzymes may require significant post-translational modifications that can only be achieved in a eukaryotic host. This will tend to limit the applicability of any eukaryotic HAS or chondroitin synthase or heparosan synthase gene that is obtained. Moreover, those of ordinary skill in the art will likely realize additional advantages in terms of time and ease of genetic manipulation where a prokaryotic enzyme gene is sought to be employed. These additional advantages include (a) the ease of isolation of a prokaryotic gene because of the relatively small size of the genome and, therefore, the reduced amount of screening of the corresponding genomic library and (b) the ease of manipulation because the overall size of the coding region of a prokaryotic gene is significantly smaller due to the absence of introns. Furthermore, if the product of the PmHAS-D or PmCS or PmHS1 or PmHS2 gene (i.e., the enzyme) requires posttranslational modifications, these would best be achieved in a similar prokaryotic cellular environment (host) from which the gene was derived.

Preferably, DNA sequences in accordance with the present invention will further include genetic control regions which allow the expression of the sequence in a selected recombinant host. Of course, the nature of the control region employed will generally vary depending on the particular use (e.g., cloning host) envisioned.

In particular embodiments, the invention concerns isolated DNA segments and recombinant vectors incorporating DNA sequences which encode a PmHAS-D or PmCS or PmHS1 or PmHS2 gene, that includes within its amino acid sequence an amino acid sequence in accordance with SEQ ID NO:1, 3, 5, 7, 11, 13, 14 or 15. Moreover, in other particular embodiments, the invention concerns isolated DNA segments and recombinant vectors incorporating DNA sequences which encode a gene that includes within its amino acid sequence the amino acid sequence of an HAS or chondroitin synthase or heparosan synthase gene or DNA, and in particular to an HAS or chondroitin synthase or heparosan synthase gene or cDNA, corresponding to Pasteurella multocida HAS or chondroitin synthase or heparosan synthase. For example, where the DNA segment or vector encodes a full length HAS or chondroitin synthase or heparosan synthase protein, or is intended for use in expressing the HAS or chondroitin synthase or heparosan synthase protein, preferred sequences are those which are essentially as set forth in SEQ ID NO:1, 3, 5, 7, 11, 13, 14, or 15.

Truncated PmHAS-D also falls within the definition of preferred sequences as set forth in SEQ ID NO:11. For instance, at the carboxyl terminus, approximately 270-272 amino acids may be removed from the sequence and still have a functioning HAS. Those of ordinary skill in the art would appreciate that simple amino acid removal from either end of the PmHAS-D sequence can be accomplished. The truncated versions of the sequence simply have to be checked for HAS activity in order to determine if such a truncated sequence is still capable of producing HAS.

Particular sequences that may be utilized in accordance with the presently claimed and disclosed invention were originally disclosed in detail in the parent applications listed above and previously incorporated herein by reference. The individual sequences and their corresponding SEQ ID NO's are listed in Table III. The numbering, mutations and nomenclature used in Table III to describe each of the sequences is defined in detail in the parent application, which has previously been incorporated by reference.

Nucleic acid segments having HAS or chondroitin synthase or heparosan synthase activity may be isolated by the methods described herein. The term “a sequence essentially as set forth in SEQ ID NO:X means that the sequence substantially corresponds to a portion of SEQ ID NO:X and has relatively few amino acids which are not identical to, or a biologically functional equivalent of, the amino acids of SEQ ID NO:X. The term “biologically functional equivalent” is well understood in the art and is further defined in detail herein, as a gene having a sequence essentially as set forth in SEQ ID NO:X, and that is associated with the ability of prokaryotes to produce HA or a hyaluronic acid coat or chondroitin. In the above examples “X” refers to either SEQ ID NO:1, 2, 3, 4, 5, 6, 7, 8, 9, 10, 11, 12, 13, 14, or 15.

The art is replete with examples of practitioners ability to make structural changes to a nucleic acid segment (i.e., encoding conserved or semi-conserved amino acid substitutions) and still preserve its enzymatic or functional activity. See for example: (1) Risler et al. “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (i) Ile and Val; (ii) Leu and Met, (iii) Lys, Arg, and Gln, and (iv) Tyr and Phe.”]; (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . ” Compatible changes can be made.]

These references and countless others, indicate that one of ordinary skill in the art, given a nucleic acid sequence, could make substitutions and changes to the nucleic acid sequence without changing its functionality. Also, a substituted nucleic acid segment may be highly identical and retain its enzymatic activity with regard to its unadulterated parent, and yet still fail to hybridize thereto.

The invention discloses nucleic acid segments encoding an enzymatically active HAS or chondroitin synthase or heparosan synthase from P. multocida-PmHAS and PmCS and PmHS1 and PmHS2, respectively. One of ordinary skill in the art would appreciate that substitutions can be made to the PmHAS or PmCS nucleic acid segment listed in SEQ ID NO:2 and 4 and 6 and 8, respectively, without deviating outside the scope and claims of the present invention. Standardized and accepted functionally equivalent amino acid substitutions are presented in Table IV.

TABLE III
DNA and Amino Acid Sequences Utilized in
Accordance with the Present Invention

| SEQ ID NO: | Sequence |
|---|---|
| 1 | pmHAS amino acid |
| 2 | pmHAS nucleic acid |
| 3 | pmCS amino acid |
| 4 | pmCS nucleic acid |
| 5 | pmHS1 amino acid |
| 6 | pmHS1 nucleic acid |
| 7 | pmHS2 amino acid |
| 8 | pmHS2 nucleic acid |
| 9 | potential HAS sequence motif |
| 10 | potential HAS sequence motif |
| 11 | pmHAS¹⁻⁷⁰³ (pmHAS-D) amino acid |
| 12 | pmHAS¹⁻⁷⁰³ (pmHAS-D) nucleic acid |
| 13 | pmHS1⁷⁷⁻⁶¹⁷ |
| 14 | pmCS¹⁻⁶⁹⁵ |
| 15 | pmCS⁴⁵⁻⁶⁹⁵ |

TABLE IV

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, | Glycine, Serine, Threonine, Cysteine, |
| R Groups | Asparagine, Glutamine |
| Negatively Charged R Groups | Aspartic Acid, Glutamic Acid |
| Positively Charged R Groups | Lysine, Arginine, Histidine |

Another preferred embodiment of the present invention is a purified nucleic acid segment that encodes a protein in accordance with SEQ ID NO:1, 3, 5, 7, 9, 11, 13, 14 or 15, further defined as a recombinant vector. As used herein, the term “recombinant vector” refers to a vector that has been modified to contain a nucleic acid segment that encodes an HAS or chondroitin synthase or heparosan synthase protein, or fragment thereof. The recombinant vector may be further defined as an expression vector comprising a promoter operatively linked to said HAS, CS or HS encoding nucleic acid segment.

A further preferred embodiment of the present invention is a host cell, made recombinant with a recombinant vector comprising an HAS or chondroitin synthase or heparosan synthase gene. The preferred recombinant host cell may be a prokaryotic cell. In another embodiment, the recombinant host cell is a eukaryotic cell. As used herein, the term “engineered” or “recombinant” cell is intended to refer to a cell into which a recombinant gene, such as a gene encoding HAS or chondroitin synthase or heparosan synthase, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter not naturally associated with the particular introduced gene.

In preferred embodiments, the HAS or chondroitin synthase or heparosan synthase encoding DNA segments further include DNA sequences, known in the art functionally as origins of replication or “replicons”, which allow replication of contiguous sequences by the particular host. Such origins allow the preparation of extrachromosomally localized and replicating chimeric segments or plasmids, to which HAS or chondroitin synthase or heparosan synthase DNA sequences are ligated. In more preferred instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The isolation and use of other replication origins such as the SV40, polyoma or bovine papilloma virus origins, which may be employed for cloning or expression in a number of higher organisms, are well known to those of ordinary skill in the art. In certain embodiments, the invention may thus be defined in terms of a recombinant transformation vector which includes the HAS or chondroitin synthase or heparosan synthase coding gene sequence together with an appropriate replication origin and under the control of selected control regions.

Thus, it will be appreciated by those of skill in the art that other means may be used to obtain the HAS or chondroitin synthase or heparosan synthase gene or cDNA, in light of the present disclosure. For example, polymerase chain reaction or RT-PCR produced DNA fragments may be obtained which contain full complements of genes or cDNAs from a number of sources, including other strains of Pasteurella or from eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HA synthase.

Once the DNA has been isolated it is ligated together with a selected vector. Virtually any cloning vector can be employed to realize advantages in accordance with the invention. Typical useful vectors include plasmids and phages for use in prokaryotic organisms and even viral vectors for use in eukaryotic organisms. Examples include pKK223-3, pSA3, recombinant lambda, SV40, polyoma, adenovirus, bovine papilloma virus and retroviruses. However, it is believed that particular advantages will ultimately be realized where vectors capable of replication in both Lactococcus or Bacillus strains and E. coli are employed.

Vectors such as these, exemplified by the pSA3 vector of Dao and Ferretti or the pAT19 vector of Trieu-Cuot, et al., allow one to perform clonal colony selection in an easily manipulated host such as E. coli, followed by subsequent transfer back into a food grade Lactococcus or Bacillus strain for production of HA or chondroitin or heparosan. These are benign and well studied organisms used in the production of certain foods and biotechnology products. These are advantageous in that one can augment the Lactococcus or Bacillus strain's ability to synthesize HA or chondroitin or heparosan through gene dosaging (i.e., providing extra copies of the HAS or chondroitin synthase or heparosan synthase gene by amplification) and/or inclusion of additional genes to increase the availability of HA or chondroitin or heparosan precursors. The inherent ability of a bacterium to synthesize HA or chondroitin or heparosan can also be augmented through the formation of extra copies, or amplification, of the plasmid that carries the HAS or chondroitin synthase or heparosan synthase gene. This amplification can account for up to a 10-fold increase in plasmid copy number and, therefore, the HAS or chondroitin synthase or heparosan synthase gene copy number.

Another procedure that would further augment HAS or chondroitin synthase or heparosan synthase gene copy number is the insertion of multiple copies of the gene into the plasmid. Another technique would include integrating the HAS or chondroitin synthase or heparosan synthase gene into chromosomal DNA. This extra amplification would be especially feasible, since the bacterial HAS or chondroitin synthase or heparosan synthase gene size is small. In some scenarios, the chromosomal DNA-ligated vector is employed to transfect the host that is selected for clonal screening purposes such as E. coli, through the use of a vector that is capable of expressing the inserted DNA in the chosen host.

In certain other embodiments, the invention concerns isolated DNA segments and recombinant vectors that include within their sequence a nucleic acid sequence essentially as set forth in SEQ ID NO:2, 4, 6 or 8. The term “essentially as set forth” in SEQ ID NO:2, 4, 6 or 8 is used in the same sense as described above and means that the nucleic acid sequence substantially corresponds to a portion of SEQ ID NO:2, 4, 6 or 8 and has relatively few codons which are not identical, or functionally equivalent, to the codons of SEQ ID NO:2, 4, 6 or 8. The term “functionally equivalent codon” is used herein to refer to codons that encode the same amino acid, such as the six codons for arginine or serine, as set forth in Table IV, and also refers to codons that encode biologically equivalent amino acids.

It will also be understood that amino acid and nucleic acid sequences may include additional residues, such as additional—or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and enzyme activity is concerned. The addition of terminal sequences particularly applies to nucleic acid sequences which may, for example, include various non-coding sequences flanking either of the 5′ or 3′ portions of the coding region or may include various internal sequences, which are known to occur within genes. Furthermore, residues may be removed from the N or C terminal amino acids and yet still be essentially as set forth in one of the sequences disclosed herein, so long as the sequence meets the criteria set forth above, as well.

Allowing for the degeneracy of the genetic code as well as conserved and semi-conserved substitutions, sequences which have between about 40% and about 80%; or more preferably, between about 80% and about 90%; or even more preferably, between about 90% and about 99%; of nucleotides which are identical to the nucleotides of SEQ ID NO:2, 4, 6 or 8 will be sequences which are “essentially as set forth” in SEQ ID NO:2, 4, 6 or 8. Sequences which are essentially the same as those set forth in SEQ ID NO:2, 4, 6 or 8 may also be functionally defined as sequences which are capable of hybridizing to a nucleic acid segment containing the complement of SEQ ID NO:2, 4, 6 or 8 under “standard stringent hybridization conditions”, “moderately stringent hybridization conditions,” “less stringent hybridization conditions,” or “low stringency hybridization conditions.” Suitable standard” or “less stringent” hybridization conditions will be well known to those of skill in the art and are clearly set forth hereinbelow. In a preferred embodiment, standard stringent hybridization conditions or less stringent hybridization conditions are utilized.

The terms “standard stringent hybridization conditions,” “moderately stringent conditions,” and “less stringent hybridization conditions” or “low stringency hybridization conditions” are used herein, describe those conditions under which substantially complementary nucleic acid segments will form standard Watson-Crick base-pairing and thus “hybridize” to one another. A number of factors are known that determine the specificity of binding or hybridization, such as pH; temperature; salt concentration; the presence of agents, such as formamide and dimethyl sulfoxide; the length of the segments that are hybridizing; and the like. There are various protocols for standard hybridization experiments. Depending on the relative similarity of the target DNA and the probe or query DNA, then the hybridization is performed under stringent, moderate, or under low or less stringent conditions.

The hybridizing portion of the hybridizing nucleic acids is typically at least about 14 nucleotides in length, and preferably between about 14 and about 100 nucleotides in length. The hybridizing portion of the hybridizing nucleic acid is at least about 60%, e.g., at least about 80% or at least about 90%, identical to a portion or all of a nucleic acid sequence encoding a HAS or chondroitin or heparin synthase or its complement, such as SEQ ID NO:2, 4, 6 or 8 or the complement thereof. Hybridization of the oligonucleotide probe to a nucleic acid sample typically is performed under standard or stringent hybridization conditions. Nucleic acid duplex or hybrid stability is expressed as the melting temperature or Tₘ, which is the temperature at which a probe nucleic acid sequence dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC, SSPE, or HPB). Then, assuming that 1% mismatching results in a 1° C. decrease in the Tₘ, the temperature of the final wash in the hybridization reaction is reduced accordingly (for example, if sequences having >95% identity with the probe are sought, the final wash temperature is decreased by about 5° C.). In practice, the change in Tₘ can be between about 0.5° C. and about 1.5° C. per 1% mismatch. Examples of standard stringent hybridization conditions include hybridizing at about 68° C. in 5×SSC/5× Denhardt's solution/1.0% SDS, followed with washing in 0.2×SSC/0.1% SDS at room temperature or hybridizing in 1.8×HPB at about 30° C. to about 45° C. followed by washing a 0.2-0.5×HPB at about 45° C. Moderately stringent conditions include hybridizing as described above in 5×SSC\5× Denhardt's solution 1% SDS washing in 3×SSC at 42° C. The parameters of salt concentration and temperature can be varied to achieve the optimal level of identity between the probe and the target nucleic acid. Additional guidance regarding such conditions is readily available in the art, for example, by Sambrook et al., 1989, Molecular Cloning, A Laboratory Manual, (Cold Spring Harbor Press, N.Y.); and Ausubel et al. (eds.), 1995, Current Protocols in Molecular Biology, (John Wiley & Sons, N.Y.). Several examples of low stringency protocols include: (A) hybridizing in 5×SSC, 5× Denhardts reagent, 30% formamide at about 30° C. for about 20 hours followed by washing twice in 2×SSC, 0.1% SDS at about 30° C. for about 15 min followed by 0.5×SSC, 0.1% SDS at about 30° C. for about 30 min (FEMS Microbiology Letters, 2000, vol. 193, p. 99-103); (B) hybridizing in 5×SSC at about 45° C. overnight followed by washing with 2×SSC, then by 0.7×SSC at about 55° C. (J. Viological Methods, 1990, vol. 30, p. 141-150); or (C) hybridizing in 1.8×HPB at about 30° C. to about 45° C.; followed by washing in 1×HPB at 23° C.

Naturally, the present invention also encompasses DNA segments which are complementary, or essentially complementary, to the sequences set forth in SEQ ID NO:2, 4, 6 or 8. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment of SEQ ID NO:2, 4, 6 or 8.

The nucleic acid segments of the present invention, regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

Naturally, it will also be understood that this invention is not limited to the particular amino acid and nucleic acid sequences of SEQ ID NOS:1-15. Recombinant vectors and isolated DNA segments may therefore variously include the HAS or chondroitin synthase or heparosan synthase coding regions themselves, coding regions bearing selected alterations or modifications in the basic coding region, or they may encode larger polypeptides which nevertheless include HAS or chondroitin synthase or heparosan synthase-coding regions or may encode biologically functional equivalent proteins or peptides which have variant amino acids sequences.

The DNA segments of the present invention encompass biologically functional equivalent HAS or chondroitin synthase or heparosan synthase proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the HAS or chondroitin synthase or heparosan synthase protein or to test HAS or chondroitin synthase or heparosan synthase mutants in order to examine HAS or chondroitin synthase or heparosan synthase activity at the molecular level.

Traditionally, chemical or physical treatments of polysaccharides were required to join two dissimilar materials. For example, a reactive nucleophile group of one polymer or surface was exposed to an activated acceptor group of the other material. Two main problems exist with this approach, however. First, the control of the chemical reaction cannot be refined and differences in temperature and level of activation often result in a distribution of several final products that vary from lot to lot preparation. For instance, several chains may be cross-linked in a few random, ill-defined areas and the resulting sample is not homogenous. Second, the use of chemical reactions to join molecules often leaves an unnatural or nonbiological residue at the junction of biomaterials. For example, the use of an amine and an activated carboxyl group would result in an amide linkage. This inappropriate residue buried in a carbohydrate may pose problems with biological systems such as degradation products which accumulate to toxic levels or may trigger an immune response.

Most polysaccharide polymers must be of a certain length before their physical or biological properties become apparent. Often the polysaccharide must comprise at least 20-100 sugar units. Certain enzymes that react with exogenous polymers have been previously available, but typically add only one sugar unit. The unique GAG synthase enzymes described in the present invention, PmHAS, PmCS, PmHS1 and PmHS2, can form polymers of at least 100-400 sugar units in length. The present invention thus results in long, defined linear polymers composed of natural glycosidic linkages. Also, under other conditions or catalytic formats (DeAngelis et al., 2003), the GAG synthases may add just one sugar unit or a few units to form oligosaccharides (i.e., chains of 2 to ˜20 saccharides long); therefore, the present invention thus results in shorter, defined linear polymers composed of natural glycosidic linkages.

The four known glycosaminoglycan synthesizing enzymes from Pasteurella multocida bacteria normally make polymers similar to or identical to vertebrate polymers. These bacteria employ the polysaccharide, either HA (Type A bacteria) or chondroitin (Type F bacteria) or heparosan (Type D), as an extracellular coating to serve as molecular camouflage. Native enzymes normally make polymer chains of a single type of sugar repeat. If a recombinant HA synthase enzyme is employed, however, the enzyme can be forced to work on an exogenous acceptor molecule. For instance, the recombinant enzyme may be incubated with a polymer acceptor and the recombinant enzyme will then elongate the acceptor with UDP-sugar precursors. The known native enzymes do not perform this reaction since they already contain a growing polymer chain.

PmHAS, a 972 amino acid residue protein from Pasteurella multocida, is made in recombinant Escherichia coli. Other functional derivatives of PmHAS, for example an enzyme called PmHAS-D, have been produced which are soluble. The soluble form can be prepared in larger quantities and in a purer state than the naturally-occurring full-length enzyme. The preferred E. coli strains do not have an UDP-Glc dehydrogenase and therefore the recombinant enzyme does not make a HA chain in the foreign host. Therefore the enzyme is in a “virgin” state since the empty acceptor site can be occupied with foreign polymers. For example, the recombinant enzyme may be incubated in a mixture containing 50 mM Tris pH 7.2; 0.1-20 mM MnCl₂; ˜0.1-50 mM UDP-GlcA; ˜0.1-50 mM UDP-GlcNAc; and a suitable acceptor at 30° C. for 30-180 minutes. Suitable acceptors can be short HA chains (two or more sugar units) or short chondroitin sulfate chains (5 sugar units) or long chondroitin sulfate chains (˜10² sugar units). In the case of the latter two acceptors, the PmHAS, and its derivatives, then elongates the foreign acceptors (i.e., long or short chondroitin oligosaccharides) at their nonreducing termini with authentic HA chains of up to 400 sugars. The length of the HA chain added onto the acceptor is controlled by altering the concentration of UDP-sugars, the reaction stoichiometry of the acceptor to UDP-sugars, and/or the reaction time. Immobilized acceptors, such as beads or other solid objects with bound acceptor oligosaccharides, can also be extended by the PmHAS enzyme using UDP-sugars. In this manner, the PmHAS enzyme can be used to attach polysaccharide chains to any suitable acceptor molecule. The suitable acceptor molecule may be a native GAG oligosaccharide or, as in the presently disclosed and claimed invention, an artificial GAG mimic. Such an acceptor can be used for production of free GAG chains or GAG chains attached to a substrate, such as but not limited to, a drug.

Type A P. multocida produces a HA capsule [GlcUA-GlcNAc repeats] and possesses the PmHAS enzyme. On the other hand, Type F P. multocida produce a chondroitin or chondroitin-like polymer capsule [GlcUA-GalNAc repeats]. The DNA encoding an open reading frame (GenBank accession #AF195517) that is 87% identical to PmHAS at the protein level has been cloned; this new enzyme is called PmCS, the P. multocida chondroitin synthase. The amino acid sequence of PmCS is set forth in Seq ID NO: 3 and the PmCS nucleotide sequence is set forth in SEQ ID NO: 4. As the PmCS enzyme's sequence is so similar to PmHAS, one of ordinary skill in the art would be able to manipulate the PmCS in the same manner as that for PmHAS and any manipulation that was successful with regard to the PmHAS would be performable with the PmCS, with the exception that chondroitin chains would be grafted instead of HA. Either HA or chondroitin chains can serve as acceptors for PmCS as both acceptors serve well for PmHAS.

Such a hybrid polysaccharide material composed of both HA and chondroitin cannot be formed by any other existing process without (1) leaving unnatural residues and/or (2) producing undesirable crosslinking reactions. The hybrid polysaccharide material can serve as a biocompatible molecular glue for cell/cell interactions in artificial tissues or organs and the HA/chondroitin hybrid mimics natural proteoglycans that normally contain an additional protein intermediate between polymer chains. The present invention, therefore, obviates the requirement for a protein intermediary. A recombinant HA/chondroitin hybrid polysaccharide, devoid of such an intermediary protein, is desirous since molecules from animal sources are potentially immunogenic—the hybrid polysaccharide, however, would not appear as “foreign” to the host, thus no immune response is generated.

An intrinsic and essential feature of polysaccharide synthesis is the repetitive addition of sugar monomer units to the growing polymer. The glycosyltransferase is expected to remain in association with the nascent chain. This feature is particularly relevant for HA biosynthesis as the HA polysaccharide product, in all known cases, is transported out of the cell; if the polymer was released, then the HAS would not have another chance to elongate that particular molecule. Three possible mechanisms for maintaining the growing polymer chain at the active site of the enzyme are immediately obvious. First, the enzyme possesses a carbohydrate polymer binding pocket or cleft. Second, the nascent chain is covalently attached to the enzyme during its synthesis. Third, the enzyme binds to the nucleotide base or the lipid moiety of the precursor while the nascent polymer chain is still covalently attached.

The HAS activity of the native PmHAS enzyme found in P. multocida membrane preparations is not stimulated by the addition of HA oligosaccharides; theoretically, the endogenous nascent HA chain initiated in vivo renders the exogenously supplied acceptor unnecessary. However, recombinant PmHAS produced in an E. coli strain that lacks the UDP-GlcUA precursor, and thus lacks a nascent HA chain, is able to bind and to elongate exogenous HA oligosaccharides. As mentioned above, there are three likely means for a nascent HA chain to be held at or near the active site. In the case of PmHAS, it appears that a HA-binding site exists near or at the sugar transferase catalytic site.

Defined oligosaccharides that vary in size and composition are used to discern the nature of the interaction between PmHAS and the sugar chain. For example, it appears that the putative HA-polymer binding pocket of PmHAS will bind and elongate at least an intact HA trisaccharide (reduced tetramer). The monosaccharides GlcUA or GlcNAc alone, however, even in combination at high concentration, are not effective acceptors. Oligosaccharide binding to PmHAS appears to be somewhat selective because the heparosan pentamer, which only differs in the glycosidic linkages from HA-derived oligosaccharides, does not serve as an acceptor. However, chondroitin [GlcUA-GalNAc repeat] does serve as an acceptor for PmHAS.

Recombinant PmHAS adds single monosaccharides in a sequential fashion to the nonreducing termini of the nascent HA chain. Elongation of HA polymers containing hundreds of sugars has been demonstrated in vitro. The simultaneous formation of the disaccharide repeat unit is not necessary for generating the alternating structure of the HA molecule. The intrinsic specificity and fidelity of each half-reaction (e.g., GlcUA added to a GlcNAc residue or vice versa) apparently is sufficient to synthesize authentic HA chains.

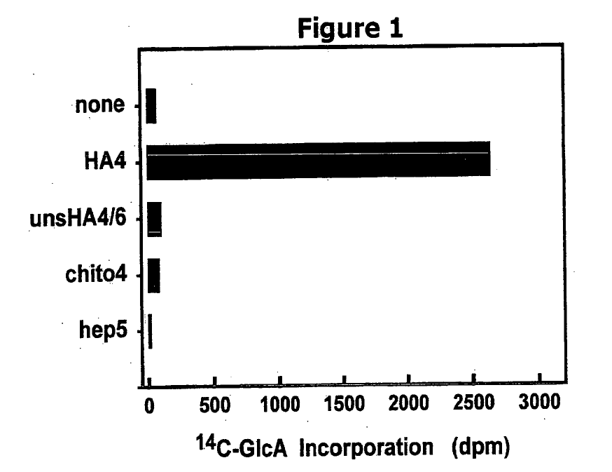

As stated above, membrane preparations from recombinant E. coli containing a PmHAS protein had HA synthase activity as judged by incorporation of radiolabel from UDP-[¹⁴C]GlcUA into polymer when co-incubated with both UDP-GlcNAc and Mn ion. Due to the similarity at the amino acid level of PmHAS to several lipopolysaccharide transferases, it was hypothesized that HA oligosaccharides serve as acceptors for GlcUA and GlcNAc transfer. Addition of unlabeled even-numbered HA tetramer (from testicular hyaluronidase digests) to reaction mixtures with recombinant PmHAS stimulates incorporation of radiolabel from UDP-[¹⁴C]GlcUA into HA polymer by ˜20- to 60-fold in comparison to reactions without oligosaccharides as shown in FIG. 1.

In FIG. 1, a series of reactions containing PmHAS (30 μg total membrane protein) were incubated with UDP-[¹⁴C]GlcUA (2×10⁴ dpm, 120 μM) and UDP-GlcNAc (450 μM) in assay buffer (50 μl reaction vol) in the presence of no added sugar (none) or various oligosaccharides (HA4, 4 μg HA tetramer; unsHA4/6, 4 μg unsaturated HA tetramer and hexamer; chito4, 50 μg chitotetraose; hep5, 20 μg heparosan pentamer). After 1 hour, the reactions were analyzed by descending paper chromatography. Incorporation of radiolabel from UDP-[¹⁴C]GlcUA into high molecular weight HA is shown. Only intact tetramer (HA4) served as an acceptor. Reactions with heparosan and chitooligosaccharides, as well as GlcNAc and/or GlcUA (not shown), incorporated as much radiolabel as parallel reactions with no acceptor. The free monosaccharides GlcUA and GlcNAc, either singly or in combination at concentrations of up to 100 μM, do not serve as acceptors; likewise, the beta-methyl glycosides of these sugars do not stimulate HAS activity.

In the same manner, PmHAS has been shown to add sugars onto a chondroitin pentamer acceptor. The PmHAS and reagents were prepared in the same manner as shown in FIG. 1, except that a chondroitin pentamer was used as the acceptor molecule. The results of this experiment are shown in TABLE V.

TABLE V

| | | Incorporation of |
|---|---|---|
| Sugar | mass | ¹⁴C-GlcUA dpm |
| none | — | 60 |
| HA₄ | 5 μg | 2,390 |
| Chondroitin Pentamer | 20 μg | 6,690 |

Thus, it can be seen that the PmHAS can utilize numerous acceptors or primer molecules as the basis for forming a polysaccharide polymer chain.

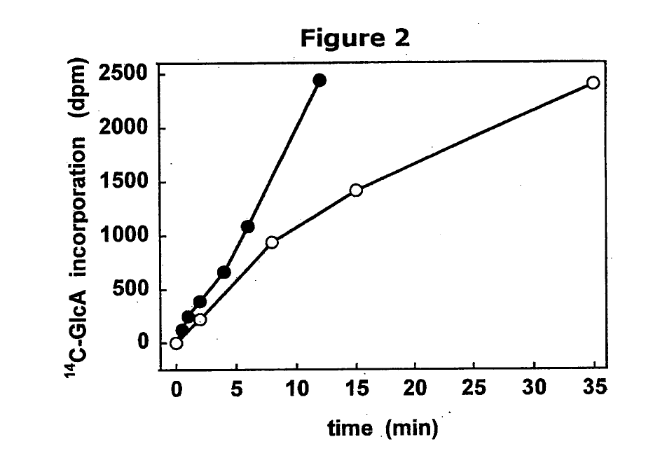

The activity of recombinant PmHAS is dependent on the simultaneous incubation with both UDP-sugar precursors and a Mn²⁺ ion. The level of incorporation is dependent on protein concentration, on HA oligosaccharide concentration, and on incubation time as shown in FIG. 2. In FIG. 2, two parallel reactions containing PmHAS with even-numbered HA oligosaccharides (105 μg membrane protein/point with a mixture of HA hexamer, octamer, and decamer, 4.4. μg total; solid circles) or six-fold more PmHAS without oligosaccharide acceptor (630 μg protein/point; open circles) were compared. The enzyme preparations were added to prewarmed reaction mixtures containing UDP-[¹⁴C]GlcUA (240 μM 6×10⁴ dpm/point) and UDP-GlcNAc (600 μM) in assay buffer. At various times, 50 μl aliquots were withdrawn, terminated, and analyzed by paper chromatography. The exogenously supplied acceptor accelerated the bulk incorporation of sugar precursor into polymer product by PmHAS, but the acceptor was not absolutely required.

HA synthesized in the presence or the absence of HA oligosaccharides is sensitive to HA lyase (>95% destroyed) and has a molecular weight of ˜1-5×10⁴ Da (˜50-250 monosaccharides). No requirement for a lipid-linked intermediate was observed as neither bacitracin (0.5 mg/ml) nor tunicamycin (0.2 mg/ml) alter the level of incorporation in comparison to parallel reactions with no inhibitor.

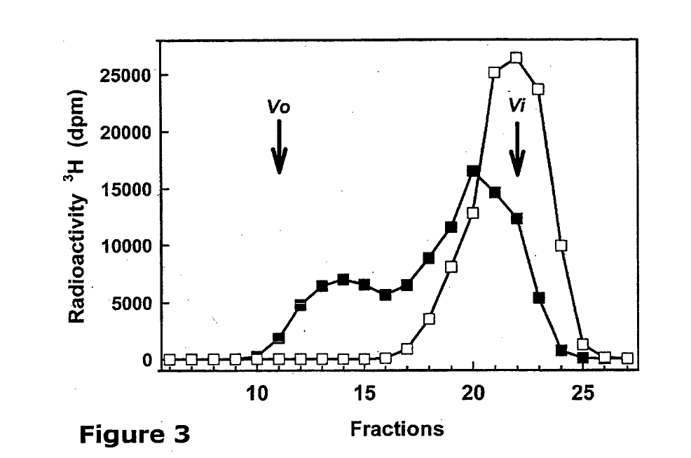

Gel filtration chromatography analysis of reactions containing recombinant PmHAS, ³H-HA tetramer, UDP-GlcNAc and UDP-GlcUA show that labeled polymers from ˜0.5 to 5×10⁴ Da (25-250 monosaccharides) are made as shown in FIG. 3. In FIG. 3, gel filtration analysis on Sephacryl S-200 (20 ml column, 0.7 ml fractions) shows that PmHAS-D makes HA polysaccharide using HA tetramer acceptor and UDP-sugars. Dextrans of greater than or equal to 80 kDa (−400 monosaccharides) elute in the void volume (Vo arrow). The starting tetramer elutes in the included volume (Vi arrow). Membranes (190 μg total protein), UDP-GlcUA (200 μM), UDP-GlcNAc (600 μM), and radiolabeled ³H-HA tetramer (1.1×10⁵ dpm) were incubated for 3 hours before gel filtration (solid squares). As a negative control, a parallel reaction containing all the components except for UDP-GlcNAc was analyzed (open squares). The small primer was elongated into higher molecular weight product if both precursors were supplied. In a parallel reaction without UDP-GlcNAc, the elution profile of the labeled tetramer is not altered.

The activity of the native PmHAS from P. multocida membranes, however, is not stimulated by the addition of HA oligosaccharides under similar conditions. The native PmHAS enzyme has an attached or bound nascent HA chain that is initiated in the bacterium prior to membrane isolation. The recombinant enzyme, on the other hand, lacks such a nascent HA chain since the E. coli host does not produce the UDP-GlcUA precursor needed to make HA polysaccharide. Therefore, the exogenous HA-derived oligosaccharide has access to the active site of PmHAS and can be elongated.

The tetramer from bovine testicular hyaluronidase digests of HA terminates at the nonreducing end with a GlcUA residue and this molecule served as an acceptor for HA elongation by PmHAS. On the other hand, the Atetramer and Δhexamer oligosaccharides produced by the action of Streptomyces HA lyase did not stimulate HA polymerization as shown in FIG. 1; unsHA4/6”. As a result of the lyase eliminative cleavage, the terminal unsaturated sugar is missing the C4 hydroxyl of GlcUA which would normally be extended by the HA synthase. The lack of subsequent polymerization onto this terminal unsaturated sugar is analogous to the case of dideoxynucleotides causing chain termination if present during DNA synthesis. A closed pyranose ring at the reducing terminus was not required by PmHAS since reduction with borohydride did not affect the HA tetramer's ability to serve as an acceptor thus allowing the use of borotritide labeling to monitor the fate of oligosaccharides.

Neither recombinant Group A HasA nor recombinant DG42 produced elongated HA-derived oligosaccharides into larger polymers in yeast. First, the addition of HA tetramer (or a series of longer oligosaccharides) did not significantly stimulate nor inhibit the incorporation of radiolabeled UDP-sugar precursors into HA (˜±5% of control value). In parallel experiments, the HAS activity of HasA or DG42 was not affected by the addition of chitin-derived oligosaccharides. Second, the recombinant enzymes did not elongate the radiolabeled HA tetramer in the presence of UDP-sugars (Table VI). These same preparations of enzymes, however, were highly active in the conventional HAS assay in which radiolabeled UDP-sugars were polymerized into HA.

As shown in Table VI, the various recombinant enzymes were tested for their ability to convert HA tetramer into molecular weight products. The reactions contained radiolabeled HA tetramer (5-8×10⁵ dpm), 750 μM UDP-GlcNAc, 360 μM UDP-GlcUA, 20 mM XCl₂, 50 mM Tris, pH 7-7.6 (the respective X cation and pH values used for each enzyme were: PmHAS, Mn/7.2; Xenopous DG42, Mg/7.6; Group A streptococcal HasA, Mg/7.0), and enzyme (units/reaction listed). As a control, parallel reactions in which the metal ion was chelated (22 mM ethylenediaminetetraacetic acid final; EDTA column, rows with +) were tested; without free metal ion, the HAS enzymes do not catalyze polymerization. After 1 hour incubation, the reactions were terminated and subjected to descending paper chromatography. Only PmHAS-D could elongate HA tetramer even though all three membrane preparations were very active in the conventional HAS assay (incorporation of [¹⁴C]GlcUA from UDP-GlcUA into polymer when supplied UDP-GlcNAc).

TABLE VI

| | | | Incorporation of HA4 into |
|---|---|---|---|
| Enzyme | Units^{a} | EDTA | polymer (pmoles) |
| PmHAS | 6^{b} | − | 240 |
| | | + | 1.7 |
| HasA | 9,800 | − | ≦0.2 |
| | | + | ≦0.2 |
| DG42 | 11,500 | − | ≦0.1 |
| | | + | ≦0.3 |

^{a}pmoles of GlcUA transfer/hr in the conventional HAS assay
^{b}measured without HA tetramer; 360 units with 100 μM HA tetramer.

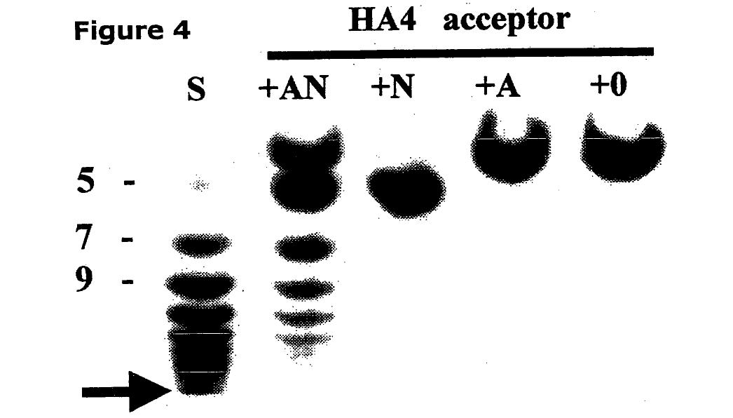

Thin layer chromatography was utilized to monitor the PmHAS-catalyzed elongation reactions containing ³H-labeled oligosaccharides and various combinations of UDP-sugar nucleotides. FIG. 4 demonstrates that PmHAS elongated the HA-derived tetramer by a single sugar unit if the next appropriate UDP-sugar precursor was available in the reaction mixture. GlcNAc derived from UDP-GlcNAc was added onto the GlcUA residue at the nonreducing terminus of the tetramer acceptor to form a pentamer. On the other hand, inclusion of only UDP-GlcUA did not alter the mobility of the oligosaccharide. If both HA precursors are supplied, various longer products are made. In parallel reactions, control membranes prepared from host cells with a vector plasmid did not alter the mobility of the radiolabeled HA tetramer under any circumstances. In similar analyses monitored by TLC, PmHAS did not utilize labeled chitopentaose as an acceptor.

As shown in FIG. 4, PmHAS extended an HA tetramer. In FIG. 4, radiolabeled HA tetramer (HA4 8×10³ dpm ³H) with a GlcUA at the nonreducing terminus was incubated with various combinations of UDP-sugars (A, 360 μM UDP-GlcUA; N, 750 μM UDP-GlcNAc; 0, no UDP-sugar), and PmHAS (55 μg membrane protein) in assay buffer for 60 minutes. The reactions (7 μl total) were terminated by heating at 95° Celsius for 1 minute and clarified by centrifugation. Portions (2.5 μl) of the supernatant were spotted onto the application zone of a silica TLC plate and developed with solvent (1.25:1:1 butanol/acetic acid/water). The beginning of the analytical layer is marked by an arrow. The positions of odd-numbered HA oligosaccharides (S lane) are marked as number of monosaccharide units. This autoradiogram (4 day exposure) shows the single addition of a GlcNAc sugar onto the HA tetramer acceptor to form a pentamer when only the subsequent precursor is supplied (N). The mobility of the labeled tetramer is unchanged if only the inappropriate precursor, UDP-GlcUA (A), or no UDP-sugar (0) is present. If both UDP-sugars are supplied, then a ladder of products with sizes of 5, 7, 9, 11, and 13 sugars is formed (+AN). In a parallel experiment, chitopentaose (8×10⁴ dpm ³H) was tested as an acceptor substrate. Under no condition was this structurally related molecule extended by PmHAS.

The present invention also demonstrates that small mimics of authentic GAG polymers are recognized and elongated by the GAG synthases of Pasteurella multocida. In general, enzymological studies of glycosyltransferases have focused on the catalytic residues, the donor binding site, and the acceptor binding site. Structural information on some “simple” glycosyltransferases that add only one sugar to a glycoconjugate has been obtained, but a structure has not been determined for a dual-action enzyme or a polysaccharide synthase. PmHAS and PmCS each appear to possess an independent hexosamine donor transfer site and a glucuronic acid donor transfer site, but the nature and the number of sugar acceptor sites are not known. As a first step to analyze the acceptor site(s), a range of acceptor sugars that PmHAS will elongate with the HA chain were tested, and it appears that the size of the synthase acceptor binding pocket corresponds roughly to the size of the smallest high efficiency substrate. These findings have allowed the determination of the optimal length of the sugar polymer necessary for efficient chain elongation and to design a synthetic, artificial acceptor for the synthase enzyme. In addition, this work has provided indirect evidence for two distinct acceptor sites in PmHAS.

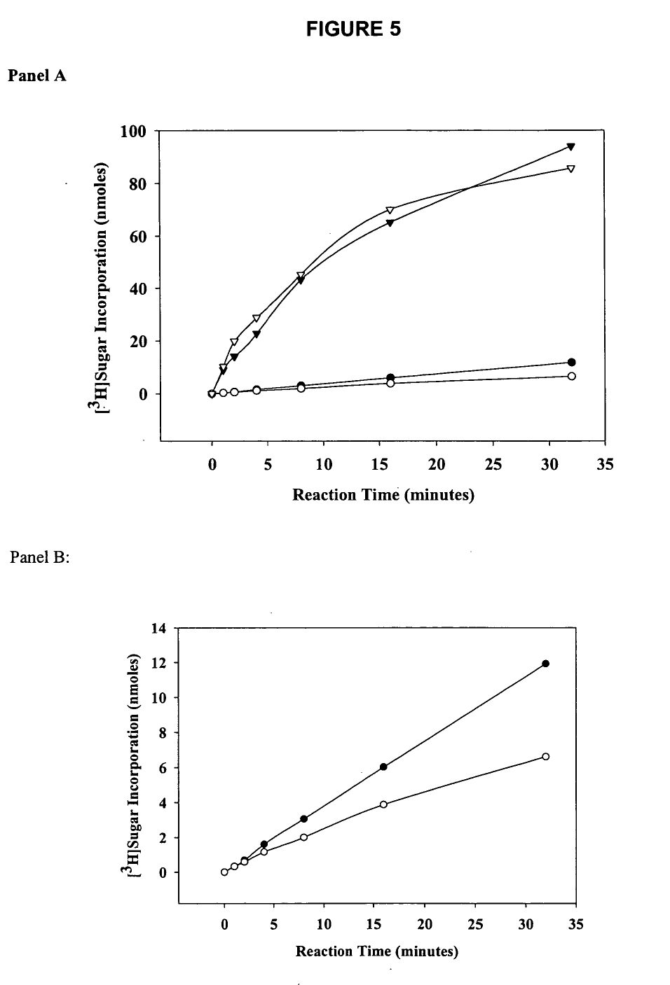

PmHAS has been shown previously to possess two relatively independent glycosyltransferase activities (UDP-N-acetylglucosamine, glucuronic acidyl: β(1,4)N-acetylglucosaminyl transferase=GlcA-transferase and UDP-glucuronic acid, N-acetylglucosaminyl: β(1,3)glucuronic acidyltransferase=GlcNAc-transferase) within a single polypeptide chain (Jing et al., 2000a and b). In order to directly compare the catalytic activities of the PmHAS GlcNAc-transferase site and GlcA-transferase site, parallel time course experiments monitoring the two single sugar addition reactions were performed. For GlcNAc-transferase activity, HA22, an acceptor which possesses a GlcA at the non-reducing terminus, was employed with UDP-[³H]GlcNAc donor. HA₂₁, an acceptor which terminates in GlcNAc, was utilized with UDP-[³H]GlcA donor for the GlcA-transferase activity. Portions of the reactions were removed at various times, quenched, and analyzed by descending paper chromatography (FIG. 5). The rate of each transferase activity corresponds to the slope (average ΔV/Δtime) at the initial phase of the reaction. The initial velocity of the GlcA-transferase activity (6.5 nmol/min) is much more rapid (˜20-fold) than the GlcNAc-transferase activity (0.32 nmol/min).

Although there is information regarding the PmHAS UDP-sugar donor-binding sites, the number of acceptor binding sites was not known and there is no precedent in the literature on any dual-action glycosyltransferases. A series of competition assays were performed to detect the presence of a single or multiple acceptor binding sites within the PmHAS polypeptide. In these reactions, one oligosaccharide (HA₁₄ or HA₁₅) served as the acceptor for the appropriate radiolabeled UDP-sugar (UDP-GlcNAc or UDP-GlcA for even-length or odd-length HA polymers, respectively). The potential competitor oligosaccharide, which is incapable of being extended due to the lack of the appropriate UDP-sugar in the reaction, was introduced into the reaction at equimolar or 10-fold higher concentrations. For example, the PmHAS GlcNAc-transferase activity was measured for (i) HA₁₄ alone (defined as ‘100% activity’), (ii) 1:1 HA₁₄ to HA₁₅ (a potential competitor which ends in a GlcNAc and therefore cannot be extended), and (iii) 1:10 HA₁₄ to HA₁₅. Conversely, the PmHAS GlcA-transferase activity was measured for (i) HA₁₅ alone (again ‘100% activity’), (ii)1:1 HA₁, to HA₁₄, and (iii)1:10 HA₁₅ to HA₁₄. Essentially, in these reactions the competitor oligosaccharide could potentially bind to an acceptor site, but elongation by the supplied UDP-sugar is impossible. In Table VII, the lack of inhibition by the oligosaccharide with the inappropriate non-reducing termini suggests that PmHAS possesses at least two independent acceptor binding sites.

To determine the minimal acceptor structure necessary for efficient HA elongation, two series of authentic, synthetic hyaluronan ([β4GlcA-β3GlcNAc]ₙ=[AN]ₙ or [β3GlcNAc-β4GlcA]ₙ=[NA]ₙ) oligosaccharides containing a methoxyphenol (MP) group at the reducing termini were investigated. The use of sugars with (a) a non-reducing termini ending in GlcA or (b) a non-reducing termini ending in GlcNAc allow the probing of both putative acceptor sites in the model. The relative activity of the methoxyphenol sugars including AN-MP, ANA-MP, ANAN-MP, ANANAN-MP, N-MP, NA-MP, NAN-MP, NANA-MP, and NANAN-MP were tested. The hydrophobicity of the methoxyphenol group of these HA-related oligosaccharides permits the use of solid phase extraction with a reverse phase sorbent for facile analysis.

TABLE VII
Competition Studies of GlcA- or GlcNAc-terminated
acceptors with PmHAS.

| | | | | GlcNAc- | GlcA- |
|---|---|---|---|---|---|
| | Accep- | Compet- | | Transferase | Transferase |
| Experiment | tor | itor | Ratio | Activity | Activity |
| I | HA₁₄ | None | — | 100% | ND |
| | HA₁₄ | HA₁₅ | 1:1 | 110% | ND |
| | HA₁₄ | HA₁₅ | 1:10 | 150% | ND |
| | HA₁₅ | None | — | ND | 100% |
| | HA₁₅ | HA₁₄ | 1:1 | ND | 93% |
| | HA₁₅ | HA₁₄ | 1:10 | ND | 99% |
| II | HA₁₄ | None | — | 100% | ND |
| | HA₁₄ | HA₁₅ | 1:1 | 140% | ND |
| | HA₁₄ | HA₁₅ | 1:10 | 160% | ND |
| | HA₁₅ | None | — | ND | 100% |
| | HA₁₅ | HA₁₄ | 1:1 | ND | 98% |
| | HA₁₅ | HA₁₄ | 1:10 | ND | 82% |
| Single sugar addition assays where one oligosaccharide served as the acceptor (e.g., [GlcA-GlcNAc]₁₄ = HA₁₄ in reaction with UDP-GlcNAc) while the other oligosaccharide (e.g., GlcNAc-[GlcA-GlcNAc]₁₄ = HA₁₅) | | | | | |
| # served as a potential competitor that may bind, but cannot be elongated. Averaged data is shown from two independent experiments. The absence of inhibition by the second oligosaccharide suggests that at least two distinct acceptor binding sites exist for the PmHAS enzyme. (ND, not done) | | | | | |

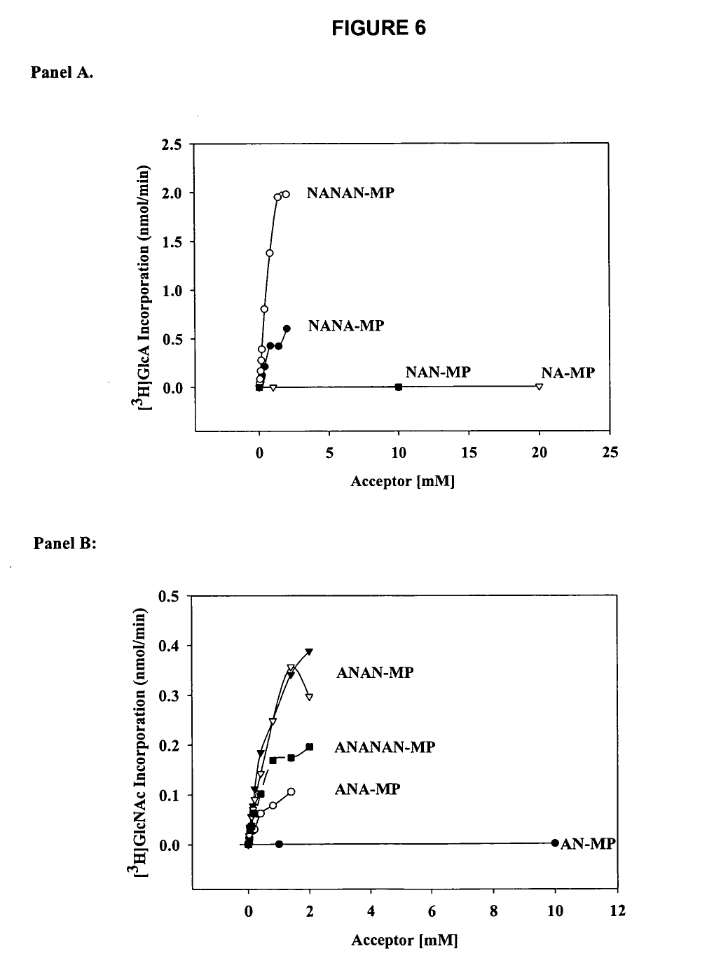

FIG. 6 depicts a uniform, representative data set of all the methoxyphenol sugars from two independent experiments. For the GlcA-transferase activity, the tetrasaccharide NANA-MP and longer served as efficient acceptors for PmHAS-catalyzed elongation (i.e., rapid reactions [4 min] and low concentrations [1-2 mM]) (FIG. 6A). Conversely, for the GlcNAc-transferase activity, the trisaccharide ANA-MP and longer were efficient acceptors (FIG. 6B). In contrast, the two possible methoxyphenol disaccharides and NAN-MP were poor substrates that required longer times (30-60 minutes) and higher concentrations (10-30 mM) to detect sugar transfer. However, the worst acceptor, N-MP, may be elongated after extensive reactions (Table VIII); thin-layer chromatography analysis of the SPE-purified product verified that this low level of activity was indeed true sugar addition and not simply a background problem (data not shown).

Competition between GlcA-terminated and GlcNAc-terminated oligosaccharides for PmHAS-mediated elongation was not observed. Therefore, Occam's razor (i.e., the simplest explanation is usually correct) was invoked to consider the possibility that PmHAS functions by utilizing at least two independent acceptor binding sites.

The experimental model system of PmHAS described herein allows for the analysis of protein-oligosaccharide interactions indirectly. Probing the active site of PmHAS with the series of methoxyphenol sugars of different lengths and other various acceptor substrates potentially reveals information about the acceptor specificity of PmHAS in the absence of a crystal structure. The kinetic data allow the ranking of various lengths of sugar acceptors to determine the optimal length of the sugar polymer necessary for efficient PmHAS chain elongation. The PmHAS GlcA-transferase site efficiently elongates the tetrasaccharide NAN-MP at a low concentration during short incubation periods while the PmHAS GlcNAc-transferase site efficiently elongated the trisaccharide (ANA-MP). Therefore, the data presented herein demonstrates that the acceptor binding sites of PmHAS contain pockets that can bind at least 3 or 4 monosaccharides for the GlcNAc-transferase or the GlcA-transferase, respectively.

The minimal length acceptors demonstrating efficient elongation are oligosaccharides that contain the trisaccharide element ANA. The predilection for ANA-MP over NAN-MP suggests there are important contacts between the carboxylate groups of the two GlcA sugars and the acceptor binding site of PmHAS. The substantial increase in the PmHAS elongation efficiency for the A-F-A acceptor, the simple proxy for ANA-MP, in comparison to the A-F acceptor also supports the hypothesis that the two GlcA groups provide important enzyme contacts. Therefore, the results presented herein demonstrate the synthesis of better analogs (e.g., higher efficiency, less expensive, animal-free manufacture).

The data generated from the activity of the methoxyphenol sugars in elongation assays suggested a requirement of two GlcA sugars for the high efficiency acceptors and thus enzyme recognition and/or utilization. Recently, characterizing the PmHAS enzymes usage of HA-like analogs with unnatural hexosamine sugars, the hydrophobic interaction appears to be involved in binding or useage. To confirm the significance of these putative critical structural elements of acceptors for PmHAS, multiple GlcA groups and a hydrophobic moiety on the hexosamine, a variety of commercially available, synthetic analogs were tested.

TABLE VIII
PmHAS Velocities (V) for methoxyphenol
sugars and synthetic acceptors.

| Sugar | V (2 mM) nmol/min | V (10 mM) nmol/min |
|---|---|---|
| AN-MP | — | 0.0013 +/− 0.0005 |
| ANA-MP | 0.11 | — |
| ANAN-MP | 0.34 | — |
| ANANAN-MP | 0.22 | — |
| N-MP | — | 0.000055 |
| NA-MP | — | 0.0043 |
| NAN-MP | — | 0.0056 +/− 0.002 |
| NANA-MP | 0.47 +/− 0.14 | — |
| NANAN-MP | 1.8 | — |
| A-F-A | 0.091 | — |
| Single sugar addition assays were performed where the next appropriate sugar necessary for chain elongation was incorporated (e.g., AN-MP + UDP-GlcNAc). The velocities at different acceptor concentrations (2 mM or 10 mM) were compared. | | |
| # Comparison of the trisaccharide efficiencies (ANA-MP at 2 mM and NAN-MP at 10 mM) indicate that ANA-MP is much more efficient at a lower substrate concentration. PmHAS efficiency for A-F-A, the simple glycoside, is similar to ANA-MP at equivalent concentrations. | | |

A collection of hydrophobic glycosides were tested as PmHAS acceptors: A-F, A-F-A, G-F-G, GalNAc-F-GalNAc, A-Nap, A-NP, GalA-NP, A-MUM, N-MUM, Gluc-MUM, A-F₃MUM, and A-CU. Most of the substrates tested were poor PmHAS acceptors as seen by the production of no or small amounts of elongation products even after utilizing extensive reaction times and/or high concentrations. However, A-F-A generated a signal similar to ANA-MP (V=˜0.10 nmoles/min at 2 mM) (Table VIII). Although A-F elongation was detected (˜9% of the A-F-A value), the addition of the second GlcA to produce A-F-A greatly boosted the velocity.

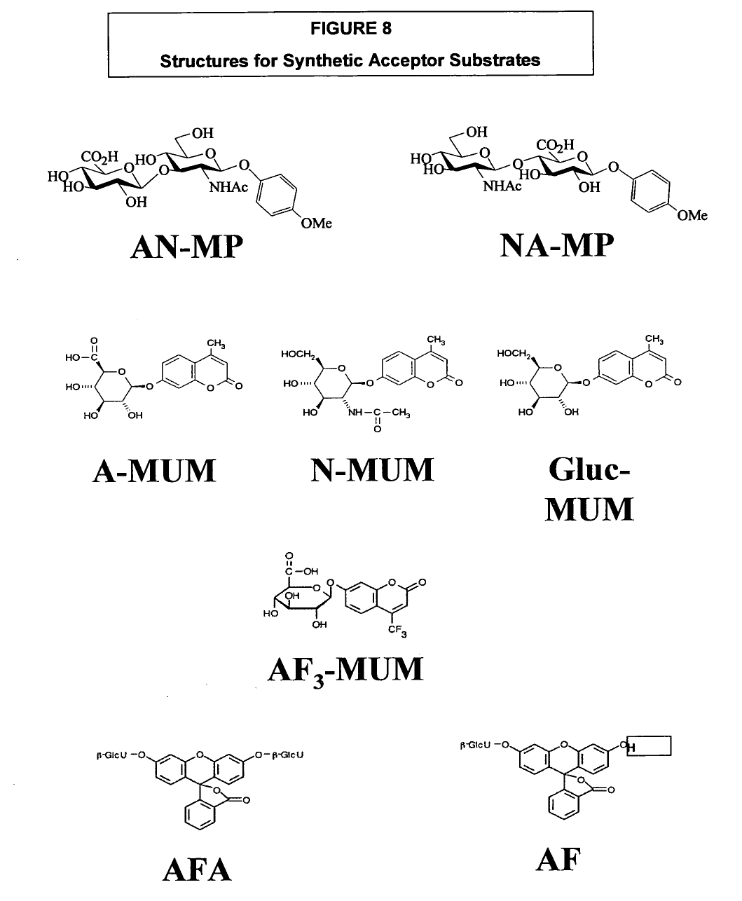

To verify that PmHAS was incorporating the GlcNAc sugar onto A-F-A, a single sugar addition reaction was analyzed by mass spectrometry (FIG. 8). After a two-hour reaction incubation period, the major reaction product was a compound formed by the addition of a single GlcNAc residue (AFA-N; experimental=683 Da; theoretical=684 Da). Upon longer incubation, PmHAS added a GlcNAc to both sides of the substrate (experimental=886 Da; theoretical=887 Da) but the single addition product was still more abundant. Thus the increase in activity of A-F-A over A-F was not due to a simple doubling of the number of usable termini. Two other structurally similar compounds, G-F-G and GalNAc-F-GalNAc, however, did not show high activity (<2%). Therefore, the substrate containing both the hydrophobic component as well as two GlcA sugars elicited the best enzyme activity, reinforcing the importance of both characteristics.

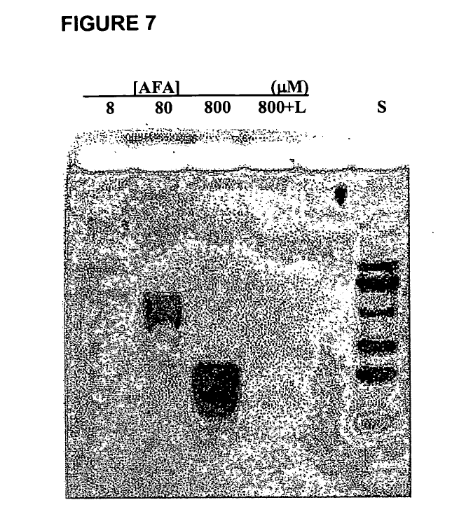

The success of A-F-A as an acceptor substrate initiated the exploration of its utility as a primer for the synthesis of monodisperse preparations of HA. An acceptor molecule will bypass the slow PmHAS initiation step resulting in synchronized reactions that yield monodisperse polymer products asin Jing et al. (2004). Polymerization reactions with A-F-A at three different concentrations (8, 80, and 800 μM) were performed and analyzed by gel electrophoresis (FIG. 7). The size of the products were ˜1,500 kDa, ˜400 kDa, or ˜175 kDa for various reactions where higher concentrations of A-F-A yielded smaller chains as expected. Essentially, the presence of a low concentration of acceptor with a finite amount of UDP-sugars will synthesize large HA products; conversely, the presence of a high concentration of acceptor and the same amount of UDP-sugars will synthesize smaller HA products (Jing et al., 2004). Gel filtration analysis with ultraviolet absorbance detection proved that the polymer contained the fluorescein aglycone (not shown). Lyase degradation of the A-F-A reaction products proved that authentic HA chains were produced.

Monodisperse HA built on A-F-A as a primer will not fluoresce until hyaluronidases remove the HA chains and β-glucuronidase cleaves the GlcA groups proximal to the fluoresceine moiety. These degradation enzymes co-exist in the lysosome thus such probes should be suitable for tracking HA degradation following uptake via receptors in liver sinusoidal cells or lymph node cells.

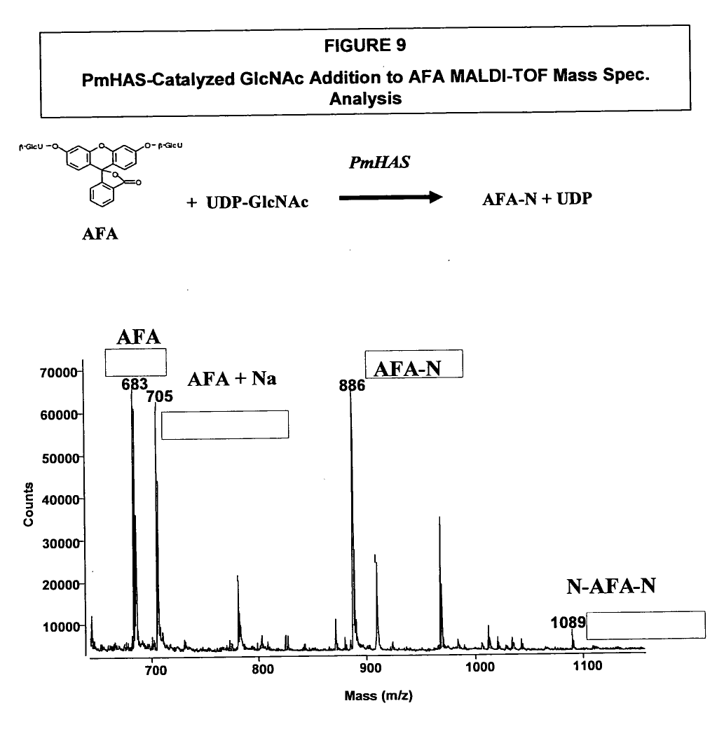

FIG. 9 illustrates the chemical structures of various candidate acceptor molecules. The AN-MP and NA-MP are sugars that precisely mimic the natural HA sugar linkage, but are not good acceptors in comparison to the previously described HA₄ (symbolically ANAN, a tetrasaccharide) due to their short length (a disaccharide); high concentrations and long times are required for the reactions, but eventually these molecules will serve as a primer for GAG synthesis. Likewise, a single GlcNAc (N) monosaccharide or a single GlcA (A) monosaccharide with an aromatic group (e.g., N-MUM, A-F, etc) are not as good acceptors as HA₄, but they do work better than the underivatized monosaccharides GlcNAc or GlcA. In the present invention, a preferred acceptor molecule is AFA. It has two GlcA groups and the aromatic nucleus that allows it to serve as a very good acceptor. Similar molecules or derivatives are expected to display good activity as acceptors for the Pasteurella GAG synthases.

Table IX demonstrates the use of AFA as an Acceptor by PmCS, the chondroitin synthase. The artificial sugar was elongated with a chondroitin chain by the PmCS enzyme, as shown by significant incorporation of radioactive sugar.

Table X demonstrates the use of AFA as an acceptor by PmHS1, the heparosan synthase. Again, the artificial sugar was elongated with a heparosan chain by the PmHS enzyme, as shown by the increased incorporation of radioactive sugars.

TABLE IX
Use of AFA as an acceptor by PmCS.

| Acceptor | [³H]GalNAc incorporation (dpm) |
|---|---|
| None | 280 |
| AFA | 14,600 |
| Two parallel 25 μl reactions in the presence or the absence of 5 mM AFA containing 1 mM UDP-GlcA, 0.15 μCi UDP-[³H]GalNAc, 35 μg of PmCS⁴⁵⁻⁶⁹⁵ enzyme in a buffer of 50 mM Tris, pH 7.2, 1 M ethylene glycol, 5 mM MnCl₂ were reacted for 1 hour at 30° C. | |
| # The polymerized reaction products were analyzed by the solid phase extraction method. | |

TABLE X
Use of AFA as an Acceptor by PmHS1.

| | [³H]GlcNAc | [¹⁴C]GlcA |
|---|---|---|
| Acceptor | incorporation (dpm) | incorporation (dpm) |
| None | 2,500 | 6,800 |
| AFA | 6,300 | 13,200 |
| heparosan | 12,800 | 38,000 |
| Three parallel 25 μl reactions with (a) no acceptor, (b) 0.07 mM AFA (0.05 μg), or (c) 6 μg of natural heparosan polymer containing 0.2 mM UDP-[¹⁴C]GlcA (˜0.1 μCi), 0.2 mM UDP-[³H]GlcNAc (˜0.1 μCi), an extract containing a fusion enzyme composed of | | |
| # thioredoxin-PmHS1 (140 μg total protein) in 50 mM Tris, pH 7.2, 10 mM MnCl₂ buffer were assembled. The mixtures were reacted for 1 hour at 30° C. The polysaccharide reaction products were analyzed by the descending paper chromatography method. | | |

Thus the three GAG synthases can utilize certain artificial acceptors or primers (e.g., AFA) that are not naturally occurring GAG sugars for the production of small GAG polymers (e.g., oligosaccharides as in DeAngelis et al., 2003) or long GAG polymers (e.g., polysaccharides as in Jing & DeAngelis, 2004). Likewise, it is expected that the artificial acceptor, if attached to an organic molecule (e.g., drug or medicament or a lipid of a liposome) or to a surface (e.g., a sensor, stent, etc.) would serve as primer for GAG extension. The main benefits of artificial primers in comparison to natural GAG acceptors include: more facile production methods; less expensive to synthesize; no use of animal-derived products (free of allergens and adventitious agents [e.g., virus, prions] and philosophical concerns [e.g., religious or cultural] and source shortfalls); simpler structures that may facilitate regulatory approval; and smaller, compact molecules with better therapeutic index or availability.

The main structural feature of the artificial acceptors is the presence of one or two monosaccharides (from the group that is found in the normal GAG composition) attached to an organic hydrophobic molecule. In a preferred case, two GlcA sugars attached to an aromatic nucleus work efficiently. Of course, the truncation/removal of non-interacting surfaces and/or the addition of more favorable surfaces to the acceptor, and/or optimizing the monosaccharide or GlcA spacing is anticipated to increase acceptor efficiency. Thus in the optimization process of the creation of artificial acceptors, a GAG mimic that does not contain intact sugar rings or saccharide structure may eventually be created.

With the advent of new biomaterials and biomimetics, hybrid polysaccharide materials will be required to serve the medical field. A major goal of bioengineering is the design of implanted artificial devices to repair or to monitor the human body. Versatile semiconductors, high-strength polymers, and durable alloys have many properties that make these materials desirable for bioengineering tasks. However, the human body has a wide range of defenses and responses that hinder the utilization of modern man-made substances. As different tissues and organs are identified as future recipients of biotechnology, it will be imperative to have an assortment of non-immunogenic polymers that can act as adhesives or protective coatings. Emulsification or adhesion industrial processes are also well suited for use with the present invention and other more suitable enzymes may be employed to graft useful molecules.

In the present invention, HA oligosaccharides and other novel primer materials are deposited onto the inorganic substrate using chemistry known to those of ordinary skill in the art and similar reaction processes. For example, a reactive epoxy surface can be made which in turn can react with amino compounds derived from HA-oligosaccharides. As shown in the present invention, artificial acceptors may also be used as primers. Once the primer materials have been deposited onto the inorganic substrate, PmHAS-D is utilized to form a protective coating of HA-polymer on the inorganic substrate. The HA polymer coating thereby protects the substrate from the body's immune system while allowing the substrate to perform an indicated purpose such as sensing, detection or drug delivery.

The majority of existing artificial materials suitable for implants and sensors, to some degree, usually (a) cause a foreign-body reaction due to the interactions with tissues or biological fluids or (b) lack substantial connectivity with the body due to their relative inertness. The HA polymer coating of the present invention overcomes these two stumbling blocks. A uniform coating of naturally occurring HA prevents an artificial components implanted into the body from spawning adverse effects such as an immune response, inappropriate clotting and/or inflammation. Furthermore, because HA is involved in maintaining the integrity of tissues and wound-healing, the HA polysaccharide coating encourages the acceptance of the artificial structure within the body.

The HA polymer attached to a biosensor acts as an external barrier protecting the sensor from the body's environment. However, in any sensing application, the chemical analyte must be able to contact the sensing material. Therefore, the HA polymer layer must allow transport of glucose to regions inside the sensor. Other molecules also exist in the blood that may interfere with the sensor response. Phase equilibrium between components in the blood and the HA polymer layer determine the local environment of the sensing layer. The transport properties of thin HA polymer layers also allow for the use of the HA polymer as a packaging material. The HA polymer outer coating allows transport of the glucose analyte in a diffusion-controlled manner while preventing biological materials from damaging the electronic device. As the HA polymer to be deposited consists of tangled, linear chains of hydrophilic sugars, glucose and other small compounds move relatively freely in the layer. On the other hand, medium to large proteins, which may foul the sensor, are excluded from the HA layer.

As stated previously, there is precedent for utilizing HA in the medical treatment of humans. Currently, HA is employed in eye surgery, joint fluid replacement, and some surgical aids. Much investigation on the use of HA to coat biomedical devices is also underway. In the previously described coating methods, HA extracted from animal or bacterial sources is typically chemically crosslinked or physically adsorbed onto a surface. Potential problems with these methodologies include: (a) immunoreaction with animal-borne contaminants and/or introduced chemical crosslinking groups, and (b) the lack of reproducibility of the coating configuration.

Due to the relative absence of foreign components or artificial moieties, no immunological problems occur. Depending on the particular application, the polymer length and the chain orientation can be controlled with precision. The polysaccharide surface coatings of the present invention improves the biocompatibility of the artificial material, lengthens the lifetime of the device in the cellular environment, and encourages natural interactions with host tissues.

With regard to surface coatings on solid materials, polyacrylamide beads have been coated with the HA polymer using PmHAS-D as the catalyst. First, aminoethyl-beads were chemically primed with HA oligosaccharide (a mixture of 4, 6, and 8 sugars long) by reductive amination. Beads, HA oligosaccharide, and 70 mM NaCNBH₄ in 0.2 M borate buffer, pH 9, were incubated at 42° C. for 2 days. The beads were washed with high and low salt buffers before use in the next step. Control beads without priming sugar or with chitopentaose [(GlcNAc)₅] were also prepared; beads without HA would not be expected to prime HA synthesis and the chitopentaose does not serve as an acceptor for PmHAS. Second, the various preparations of beads (15μ liters) were incubated with PmHAS-D (3 μg), 150 mM UDP-[³H]GlcNAc, 60 mM UDP-[¹⁴C]GlcUA, 20 mM MnCl₂, in 50 mM Tris, pH 7.2, at 30° C. for 60 min. The beads were then washed with high and low salt buffers. Radioactivity linked to beads (corresponding to the sugars) was then measured by liquid scintillation counting Table XI.

Only HA beads primed with the HA oligosaccharide and incubated with PmHAS-D incorporated the radiolabel from both UDP-sugar precursors indicating that the short HA sugar attached to the bead was elongated into a longer HA polymer by the enzyme. Thus far, no other known HA synthase possesses the desired catalytic activity to apply an HA polymer coating onto a primed substrate.

TABLE XI

| | | Bound GlcUA | Bound GlcNAc |
|---|---|---|---|
| Bead Type | Enzyme Added? | (¹⁴C dpm) | (³H dpm) |
| HA primer | yes | 990 | 1140 |
| HA primer | no | 10 | 10 |
| Chito primer | yes | 24 | 18 |
| No primer | yes | 5 | 35 |

Thus, as shown above, an authentic HA oligosaccharide primer was chemically coupled to a polyacrylamide surface and then this primer was further elongated using the PmHAS enzyme and UDP-sugars. Depending on the substrate, the reaction conditions can be optimized by one of ordinary skill in the art. For example, the mode of semiconductor modification, buffer conditions, HA elongation reaction time, and stoichiometry can be varied to take into account any single or multiple reaction variation. The resulting coatings can then be evaluated for efficacy and use.

Biomaterials also play a pivotal role in the field of tissue engineering. Biomimetic synthetic polymers have been created to elicit specific cellular functions and to direct cell-cell interactions both in implants that are initially cell-free, which may serve as matrices to conduct tissue regeneration, and in implants to support cell transplantation. Biomimetic approaches have been based on polymers endowed with bioadhesive receptor-binding peptides and mono- and oligosaccharides. These materials have been patterned in two- and three-dimensions to generate model multicellular tissue architectures, and this approach may be useful in future efforts to generate complex organizations of multiple cell types. Natural polymers have also played an important role in these efforts, and recombinant polymers that combine the beneficial aspects of natural polymers with many of the desirable features of synthetic polymers have been designed and produced. Biomaterials have been employed to conduct and accelerate otherwise naturally occurring phenomena, such as tissue regeneration in wound healing in the otherwise healthy subject; to induce cellular responses that might not be normally present, such as healing in a diseased subject or the generation of a new vascular bed to receive a subsequent cell transplant; and to block natural phenomena, such as the immune rejection of cell transplants from other species or the transmission of growth factor signals that stimulate scar formation.

Recently, the concept of bioadhesion was introduced into the pharmaceutical literature and has since stimulated much research and development both in academia and in industry. The first generation of bioadhesive drug delivery systems (BBDS) were based on so-called mucoadhesive polymers, i.e., natural or synthetic macromolecules, often already well accepted and used as pharmaceutical excipients for other purposes, which show the remarkable ability to ‘stick’ to humid or wet mucosal tissue surfaces. While these novel dosage forms were mainly expected to allow for a possible prolongation, better localization or intensified contact to mucosal tissue surfaces, it had to be realized that these goals were often not so easily accomplished, at least not by means of such relatively straightforward technology. However, although not always convincing as a “glue”, some of the mucoadhesive polymers were found to display other, possibly even more important biological activities, namely to inhibit proteolytic enzymes and/or to modulate the permeability of usually tight epithelial tissue barriers. Such features were found to be particularly useful in the context of peptide and protein drug delivery.

The primary goal of bioadhesive controlled drug delivery is to localize a delivery device within the body to enhance the drug absorption process in a site-specific manner. Bioadhesion is affected by the synergistic action of the biological environment, the properties of the polymeric controlled release device, and the presence of the drug itself. The delivery site and the device design are dictated by the drug's molecular structure and its pharmacological behavior.

For example, one embodiment of the present invention is the use of sutures or bandages with HA-chains grafted on the surface or throughout the material in combination with the fibrinogen glue. The immobilized HA does not diffuse away as in current formulations, but rather remains at the wound site to enhance and stimulate healing.

In the present invention, HA orchondroitin or heparosan chains would be the natural substitute for poly(acrylic-acid) based materials. HA is a negatively-charged polymer as is poly(acrylic-acid), but HA is a naturally occurring molecule in the vertebrate body and would not invoke an immune response like a poly(acrylic-acid) material.

The interest in realizing ‘true’ bioadhesion continues: instead of mucoadhesive polymers, plant or bacterial lectins, i.e., adhesion molecules which specifically bind to sugar moieties of the epithelial cell membrane, are now widely being investigated as drug delivery adjuvants. These second-generation bioadhesives not only provide for cellular binding, but also for subsequent endo- and transcytosis. This makes the novel, specifically bioadhesive molecules particularly interesting for the controlled delivery of DNA/RNA molecules in the context of antisense or gene therapy.

For the efficient delivery of peptides, proteins, and other biopharmaceuticals by nonparenteral routes, in particular via the gastrointestinal, or GI, tract, novel concepts are needed to overcome significant enzymatic and diffusional barriers. In this context, bioadhesion technologies offer some new perspectives. The original idea of oral bioadhesive drug delivery systems was to prolong and/or to intensify the contact between controlled-release dosage forms and the stomach or gut mucosa. However, the results obtained during the past decade using existing pharmaceutical polymers for such purposes were rather disappointing. The encountered difficulties were mainly related to the physiological peculiarities of GI mucus. Nevertheless, research in this area has also shed new light on the potential of mucoadhesive polymers. First, one important class of mucoadhesive polymers, poly(acrylic acid), could be identified as a potent inhibitor of proteolytic enzymes. Second, there is increasing evidence that the interaction between various types of bio(muco)adhesive polymers and epithelial cells has direct influence on the permeability of mucosal epithelia. Rather than being just adhesives, mucoadhesive polymers may therefore be considered as a novel class of multifunctional macromolecules with a number of desirable properties for their use as biologically active drug delivery adjuvants.

In the present invention, HA or other glycosaminoglycan polysaccharides are used. As HA is known to interact with numerous proteins (i.e., RHAMM, CD44) found throughout the healthy and diseased body, then naturally occurring adhesive interactions can be utilized to effect targeting, stabilization, or other pharmacological parameters. Similarly, chondroitin interacts with a different subset of proteins (i.e., platelet factor 4, thrombin); it is likely that this polymer will yield properties distinct from HA and widen the horizon of this technology.

In order to overcome the problems related to GI mucus and to allow longer lasting fixation within the GI lumen, bioadhesion probably may be better achieved using specific bioadhesive molecules. Ideally, these bind to surface structures of the epithelial cells themselves rather than to mucus by receptor-ligand-like interactions. Such compounds possibly can be found in the future among plant lectins, novel synthetic polymers, and bacterial or viral adhesion/invasion factors. Apart from the plain fixation of drug carriers within the GI lumen, direct bioadhesive contact to the apical cell membrane possibly can be used to induce active transport processes by membrane-derived vesicles (endo- and transcytosis). The nonspecific interaction between epithelia and some mucoadhesive polymers induces a temporary loosening of the tight intercellular junctions, which is suitable for the rapid absorption of smaller peptide drugs along the paracellular pathway. In contrast, specific endo- and transcytosis may ultimately allow the selectively enhanced transport of very large bioactive molecules (polypeptides, polysaccharides, or polynucleotides) or drug carriers across tight clusters of polarized epi- or endothelial cells, whereas the formidable barrier function of such tissues against all other solutes remains intact.

Bioadhesive systems are presently playing a major role in the medical and biological fields because of their ability to maintain a dosage form at a precise body-site for a prolonged period of time over which the active principle is progressively released. Additional uses for bioadhesives include: bioadhesives/mucoadhesives in drug delivery to the gastrointestinal tract; nanoparticles as a gastroadhesive drug delivery system; mucoadhesive buccal patches for peptide delivery; bioadhesive dosage forms for buccal/gingival administration; semisolid dosage forms as buccal bioadhesives; bioadhesive dosage forms for nasal administration; ocular bioadhesive delivery systems; nanoparticles as bioadhesive ocular drug delivery systems; and bioadhesive dosage forms for vaginal and intrauterine applications.

In this manner, the present invention contemplates a bioadhesive comprising HA produced from PmHAS. The present invention also contemplates a composition containing a bioadhesive comprising HA produced from PmHAS and an effective amount of a medicament, wherein the medicament can be entrapped or grafted directly within the HA bioadhesive or be suspended within a liposome which is entrapped or grafted within the HA bioadhesive. These compositions are especially suited to the controlled release of medicaments.

Such compositions are useful on the tissues, skin, and mucus membranes (mucosa) of an animal body, such as that of a human, to which the compositions adhere. The compositions so adhered to the mucosa, skin, or other tissue slowly release the treating agent to the contacted body area for relatively long periods of time, and cause the treating agent to be sorbed (absorbed or adsorbed) at least at the vicinity of the contacted body area. Such time periods are longer than the time of release for a similar composition that does not include the HA bioadhesive.

The treating agents useful herein are selected generally from the classes of medicinal agents and cosmetic agents. Substantially any agent of these two classes of materials that is a solid at ambient temperatures may be used in a composition or method of the present invention. Treating agents that are liquid at ambient temperatures, e.g., nitroglycerine, can be used in a composition of this invention, but are not preferred because of the difficulties presented in their formulation. The treating agent may be used singly or as a mixture of two or more such agents.

One or more adjuvants may also be included with a treating agent, and when so used, an adjuvant is included in the meaning of the phrase “treating agent” or “medicament.” Exemplary of useful adjuvants are chelating agents such as EDTA that bind calcium ions and assist in passage of medicinal agents through the mucosa and into the blood stream. Another illustrative group of adjuvants are the quaternary nitrogen-containing compounds such as benzalkonium chloride that also assist medicinal agents in passing through the mucosa and into the blood stream.

The treating agent is present in the compositions of this invention in an amount that is sufficient to prevent, cure and/or treat a condition for a desired period of time for which the composition of this invention is to be administered, and such an amount is referred herein as “an effective amount.” As is well known, particularly in the medicinal arts, effective amounts of medicinal agents vary with the particular agent involved, the condition being treated and the rate at which the composition containing the medicinal agent is eliminated from the body, as well as varying with the animal in which it is being used, and the body weight of that animal. Consequently, effective amounts of treating agents may not be defined for each agent. Thus, an effective amount is that amount which in a composition of this invention provides a sufficient amount of the treating agent to provide the requisite activity of treating agent in or on the body of the treated animal for the desired period of time, and is typically less than that amount usually used.

Inasmuch as amounts of particular treating agents in the blood stream that are suitable for treating particular conditions are generally known, as are suitable amounts of treating agents used in cosmetics, it is a relatively easy laboratory task to formulate a series of controlled release compositions of this invention containing a range of such treating agent for a particular composition of this invention.

The second principle ingredient of this embodiment of the present invention is a bioadhesive comprising an amount of hyaluronic acid (HA) from PmHAS or chondroitin from PmCS or heparosan from PmHS1 or PmHS2. Such a glycosaminoglycan bioadhesive made from a HA or chondroitin or heparosan chain directly polymerized onto a molecule with the desired pharmacological property or a HA or chondroitin or heparosan chain polymerized onto a matrix or liposome which in turn contains or binds the medicament.

Chemotherapy is an important current therapeutic tool in the treatment of cancer either as a stand-alone modality or as an adjunct to surgery and/or radiotherapy. However, due to the general mechanism of cytotoxic drug action and the nature of malignant disease, several drawbacks limit the true potential of chemotherapy. We can use polymer grafting with a GAG and a drug to create targeted drugs/delivery agents.

By adding a GAG-based targeting moiety to useful, existing chemotherapy drugs, we will improve delivery and reduce toxicity. For example, linking hyaluronan oligosaccharides to a chemotherapeutic drug will create a relatively non-toxic and soluble prodrug that will bind rather selectively to up-regulated and/or activated receptors of cancer cells. The conjugate will then be internalized and transported to the lysosome where the toxic drug is released and triggers the death of the cancer cell. In contrast, normal cells will not internalize the prodrug as readily, thus these agents should be relatively non-toxic to healthy tissues.

The modular synthetic strategy of the present invention is compatible with several classes of important existing drugs with utility for treating colon, ovarian, breast, lung, and lymphoid cancers. Furthermore, in contrast to many other targeting or delivery platforms, our GAG-based conjugate compounds will be single molecular entities that should be manufactured more reproducibly and hence more likely to pass government regulatory scrutiny. Overall, the targeting prodrug approach should improve the effectiveness of existing promising drugs for single and combination therapies by lowering toxicity, increasing effectiveness, and decreasing side effects.

Of course, adding other GAGs (e.g., heparosan or chondroitin) that have an affinity for a certain cell or tissue will allow targeting of the attached molecule. In addition to toxic payloads, the attached molecule may instead be a beneficial agent such as a vitamin, growth factor, or curative gene, etc. Alternatively, the attached molecule may be part of a binary system where two components are brought together in one location or cell for the desired effect.

### Materials and Methods

Reagents and Enzyme Preparation. All reagents were of highest grade available from either Sigma or Fisher unless otherwise noted. The soluble, truncated dual-action PmHAS¹⁻⁷⁰³ enzyme or the PmCS⁴⁵⁻⁶⁹⁵ was prepared by chromatography as described previously (DeAngelis et al., 2003a and 2003b). Briefly, the recombinant cells expressing PmHAS¹⁻⁷⁰³ were extracted with 1% (w/v) octyl thioglucoside in 1 M ethylene glycol, 50 mM Hepes, pH 7.2. The clarified extract was purified on a Toyopearl Red AF resin (Tosoh, Montgomeryville, Pa.) column. The protein was eluted with a NaCl gradient (50 mM HEPES, pH 7.2, 1 M ethylene glycol with 0-1.5 M NaCl gradient in 1 h). The peak fractions with synthase, as assessed by Coomassie blue staining of SDS-PAGE gels, were pooled and concentrated by ultrafiltration. The protein content was quantitated by the Bradford assay (Pierce, Rockford, Ill.) with a bovine serum albumin standard. The final preparations were typically ˜95% pure PmHAS or PmCS based on staining of the gels. PmHS1 was prepared in E. coli and used in the form of a soluble cell lysate without purification. This form of enzyme was a thioredoxin fusion protein prepared using the pBAD/Thio TOPO kit (Invitrogen).

Sugar Acceptor Substrates. The HA₄ tetrasaccharide (with GlCA at non-reducing terminus) was derived from exhaustive digestion of HA (streptococcal) with testicular hyaluronidase, chloroform solvent extraction, and gel chromatography on P2 resin (BioRAD, Hercules, Calif.). Longer natural HA oligosaccharides (HA₁₄, HA₁₅, HA₂₀, HA₂₁) were synthesized chemoenzymatically from HA₄ and UDP-sugars using immobilized enzyme reactors (DeAngelis et al., 2003a).

A series of HA-like oligosaccharides were synthesized by organic chemistry methodology; each sugar contained a para-methoxyphenyl group at the reducing end (Halkes et al., 1998). For simplicity, a coding scheme is used to designate each monosaccharide: GlcA, A; GlcNAc, N; methoxyphenyl, MP. For example, the compound AN-MP refers to GlcA-GlcNAc-MP as read from non-reducing end to reducing end.

Various synthetic glycosides were purchased and again the simple coding scheme was applied: fluorescein mono-β-D-glucuronide, A-F; fluorescein di-β-D-glucuronide, A-F-A (Molecular Probes, Eugene, Oreg.); fluorescein di-β-D-glucopyranoside, G-F-G (Molecular Probes); fluorescein di-β-D-N-acetylgalactosamine, GalNAc-F-GalNAc (Marker Gene Technologies, Eugene, Oreg.); 1-naphthyl β-D-glucuronide, A-NAP; p-nitrophenyl-β-D-glucuronide, A-NP (Calbiochem, La Jolla, Calif.); 4-nitrophenyl-β-D-galacturonide, GalA-NP; 4-methylumbelliferyl β-D-glucuronide, A-MUM; β-trifluoromethylumbelliferyl β-D-glucuronide, A-F3MUM (Molecular Probes); 3-carboxyumbelliferyl β-D-glucuronide, A-CU (Molecular Probes); 4-methylumbelliferyl N-acetyl-β-D-glucosaminide, N-MUM; and 4-methylumbelliferyl β-D-glucopyranoside, G-MUM.

Single Sugar Addition Assays. The assays monitored the transfer of either (a) a single GlcA to an acceptor with a non-reducing end terminating in GlcNAc according to the reaction:

n UDP-GlcA+n GlcNAc-X→n GlcA-GlcNAc-X+n UDP

or (b) a single GlcNAc to an acceptor with a non-reducing end terminating in GlcA according to the reaction:

n UDP-GlcNAc+n GlcA-X→n GlcNAc-GlcA-X+n UDP

where X=the remainder of the acceptor molecule. PmHAS (0.4 μM) was assayed in 25 μl reactions containing a titration of one of the various acceptors, 50 mM Tris, pH 7.2, 5 mM MnCl₂, 1 M ethylene glycol, and either 1 mM UDP-[³H]GlCA (0.15 μCi) or 1 mM UDP-[³H]GlcNAc (0.15 μCi) (PerkinElmer, Shelton, Conn.), respectively. Control assays without any acceptor were also performed in parallel; this background value was subtracted from the value obtained in acceptor-containing assays. Reactions were incubated at 30° C. for various times ranging from 4-1260 minutes.

Enzyme activity was linear with respect to time and the reactions consumed less than 5% of the UDP-sugar substrate. All assay points were performed in duplicate and the values were averaged. The data were plotted using the Michaelis-Menten equation (Velocity=Velocity_{MAX} [Substrate]/K_{M}+[Substrate]) with Sigma Plot software (Rockware, Golden, Colo.) where the apparent Michaelis-Menten constants (K_{M}) were derived from the concentration of sugar that yields 50% of maximal incorporation.

If the acceptor possessed a hydrophobic aglycone (e.g., MP, F, etc.), then reversed phase solid phase extraction (SPE) was employed to separate products from reactants. If native HA longer than HA₁₄ was tested, then descending paper chromatography was utilized.

For SPE analysis, reactions were terminated by placing on ice and diluted with 275 μl of ice-cold 1 M NaCl. The free unincorporated UDP-[³H] sugar precursors were separated from the elongated reaction products using reversed phase cartridges (Strata-X polymeric 33 μm resin; 30 mg sorbent/1 ml cartridge; Phenomenex, Torrance, Calif.) and a vacuum manifold. When passing solvents through the column, the bed was not allowed to dry until directly before and after the elution step. Columns were sequentially conditioned with 1 ml each of 100% methanol, 50% methanol, and water. Columns were equilibrated with 1 ml of 1 M NaCl and then samples in 1 M NaCl were added to the sorbent bed. The columns were washed with 7 ml of 1 M NaCl allowing retention of the methoxyphenol or hydrophobic compounds and the removal of UDP-[³H] sugars. After completing the NaCl wash, the column bed was air-dried for 30 seconds with vacuum suction. To release the acceptors from the sorbent, the columns were eluted with 2 ml of 50% methanol. BioSafe II cocktail (4 ml) (Research Products International, Chicago, Ill.) was added to 1 ml of the eluted sample and incorporation of the [³H] sugars was quantitated by liquid scintillation counting.

For descending paper chromatography analysis, reactions were terminated by adding SDS to a final concentration of 2% and then spotted onto strips of Whatman 3M paper and the reaction products at the origin were separated from the free UDP-[³H] sugars by development with 65:35 ethanol/1M ammonium acetate, pH 5.5 overnight (Jing et al., 2000a). The origins were cut from the paper strips and eluted in 750 μl water for 1 hour. BioSafe II cocktail (4 ml) was added and incorporation of the [³H] sugars was quantitated by liquid scintillation counting.

HA Polymerization Assays. The Pm HAS-catalyzed polymerization assay measured the incorporation of both GlcA and GlcNAc onto acceptors to form longer HA chains as in:

n UDP-GlcNAc+n UDP-GlcA+nX 4 n (GlcNAc-GlcA)ₙ—X+2n UDP or

n UDP-GlcNAc+n UDP-GlcA+nX→n (GlcA-GlcNAc)ₙ-X+2n UDP

where X=the acceptor (exact product depends on the identity of the acceptor non-reducing terminus). PmHAS polymerization activity was assayed under identical conditions as the single sugar addition assay except 1 mM UDP-[3H]GlcA (0.15 μCi) and 1 mM UDP-GlcNAc were present simultaneously. Descending paper chromatography was used to measure incorporation as described above. Experiments were performed in duplicate and data points were averaged unless otherwise noted.

Sugar Competition Assays. To assess the number of acceptor sites within the PmHAS polypeptide, competition experiments were devised between two distinct oligosaccharides each having a different nonreducing terminal sugar; HA₁₄ terminates in GlcA while HA₁₅ terminates in GlcNAc. Both oligosaccharides were present simultaneously in a reaction with only one type of radiolabeled UDP-sugar nucleotide. In this situation, one oligosaccharide served as the acceptor substrate molecule and the other as the potential competitor molecule which cannot be extended. For example, in one experiment with UDP-[³H]GlcNAc, HA₁₄ functioned as the acceptor for the addition of the GlcNAc moiety while HA₁₅ served as the potential competitor. In the converse experiment with UDP-[³H]GlcA, HA₁₅ served as the acceptor for the addition of the GlcA monosaccharide while HA₁₄ functioned as the potential competitor. The reactions without potential competitor (only the acceptor with the appropriate non-reducing terminus) were run in parallel and served as the “100% activity” value. The products of reactions were analyzed by paper chromatography.

Analysis of in vitro synthesized HA. The synthetic molecule fluorescein di-β-D-glucuronide (A-F-A) was used as the acceptor to synchronize the synthesis of monodisperse HA preparations. Reactions conditions were 50 mM Tris, pH 7.2, 5 mM MnCl₂, 1 M ethylene glycol, 12.2 mM UDP-GlcA, 12.2 mM UDP-GlcNAc, and 14 μM PmHAS plus A-F-A acceptor at three different concentrations (8 μM, 80 μM, and 800 μM) in a total volume of 25 μl. Reactions were incubated at 30° C. overnight. The size of the products was analyzed using agarose gel electrophoresis (1.2%; 1×TAE buffer (40 mM Tris acetate, 2 mM EDTA); 30 V) (Lee et al., 1994) and Stains-All dye detection (0.005% w/v in ethanol). Select-HA Lo and Hi Ladders composed of monodisperse HA polymers (Jing et al., 2004) were used as standards (Hyalose, Oklahoma City, Okla.). To assess the authenticity of the HA linkages, the reactions were treated with Streptomyces HA lyase, an enzyme that degrades no other GAG except HA. The pH for the reaction was adjusted to pH 6 by the addition of sodium acetate (50 mM final). The reaction was boiled for 1 min at 95° C. and centrifuged to remove PmHAS. After overnight incubation with Streptomyces lyase, the sample was loaded onto the agarose gel.

To ascertain the presence of the aglycone in the product polymer chains, the reactions were adjusted to 0.2 M sodium nitrate and analyzed by high performance gel filtration chromatography on a Polysep 4000 column (1 ml/min, 0.2 M sodium nitrate; Phenomenex, Torrance, Calif.) with UV absorbance detection at 272 nm for the A-F-A glycone. Fluorescent dextran standards with molecular weights of 4, 12, 50, and 580 kDa were used as calibrants (detection 490 nm).

To determine the absolute molecular masses of HA products, multi-angle laser light scattering/size-exclusion chromatography (MALLS-SEC) analysis was utilized. Polymers were separated on three tandem PL Aquagel-OH 60/60/50 (15 μm) columns (7.5×300 mm, Polymer Laboratories, Amherst, Mass.) in series. The columns were eluted with 50 mM sodium phosphate, 150 mM NaCl, pH 7, at 0.5 ml/min. MALLS analysis of the eluant was performed by a DAWN DSP Laser Photometer in series with an OPTI-LAB DSP interferometric refractometer (632.8 nm; Wyatt Technology, Santa Barbara, Calif.). The ASTRA software package was used to determine the absolute molecular mass using a dn/dc coefficient of 0.153 determined by Wyatt Technology.

The exact masses of products formed after reaction of A-F-A with PmHAS and UDP-GlcNAc were measured by mass spectrometry (MS). Matrix-assisted laser desorption ionization time-of-flight spectra were obtained using a Voyager Elite DE mass spectrometer. The sample in water (1 μl of ˜0.01 μg/μl oligosaccharide) was spotted on a target plate followed by 1 μl of matrix solution (10 mg/ml 6-aza-2-thiothymine in 50% acetonitrile, 49.9% water, 0.1% trifluoroacetic acid), mixed, then vacuum-dried. The samples were analyzed using negative ion, reflectron mode with the following parameters: acceleration, 20 kV; low mass gate, 400 Da; and delayed extraction, 200 ns.

Although the foregoing invention has been described in some detail by way of illustration and example for purposes of clarity of understanding, it will be obvious to those skilled in the art that certain changes and modifications may be practiced without departing from the spirit and scope thereof, as described in this specification and as defined in the appended claims below.

### REFERENCES

The following references, to the extent that they provide exemplary procedural or other details supplementary to those set forth herein, are specifically incorporated herein by reference in their entirety as though set forth herein particular.
1. Carter, G. R. and Annau, E. (1953) Am. J. Ver. Res. 14, 475-478.
2. Cheetham, J. C., Artymiuk, P. J., and Phillips, D. C. (1992) J Mol Biol 224, 613-628.
3. Chung, J. Y., Wilkie, I., Boyce, J. D., Townsend, K. M., Frost, A. J, Ghoddusi, M. and Adler, B. (2001) Infect. Immun. 69, 2487-2492.
4. DeAngelis, P. L., Papaconstantinou, J. and Weigel, P. H. (1993) J. Biol. Chem., 268, 19181-19184.
5. DeAngelis, P. L. and Weigel, P. H. (1994) Biochemistry, 33, 9033-9039.
6. DeAngelis, P. L., Jing, W., Drake, R. R., and Achyuthan, A. M. (1998) J Biol Chem 273, 8454-8458.
7. DeAngelis, P. L. (1999a) J Biol Chem 274, 26557-26562.
8. DeAngelis, P. L. (1999b) Cell. Molec. Life Sciences, 56, 670-682.
9. DeAngelis, P. L., and Padgett-McCue, A. J. (2000) J Biol Chem 275, 24124-24129.
10. DeAngelis, P. L. and White, C. L. (2002) J. Biol. Chem. 277: 7209-7213.
11. DeAngelis, P. L., Oatman, L. C., and Gay, D. F. (2003) J Biol Chem 278, 35199-35203.
12. DeAngelis, P. L. and White, C. L. (2004) J. Bacteriology. 186: 8529-8532.
13. Dougherty, B. A. and van de Rijn, I. (1994) J. Biol. Chem., 269, 169-175.
14. Duncan, G., McCormick, C., and Tufaro, F. (2001) J. Clin. Invest., 108, 511-516.
15. Finke, A., Bronne, D., Nikolaev, A. V., Jann, B. and Jann K. (1991) J. Bacteriol., 173, 4088-4094.
16. Ford, L. O., Johnson, L. N., Machin, P. A., Phillips, D. C., and Tjian, R. (1974) J Mol Biol 88, 349-371.
17. Griffiths, G., Cook, N. J., Gottfridson, E., Lind, T., Lidholt, K. and Roberts, I. S. (1998) J. Biol. Chem., 273, 11752-11757.
18. Halkes, K. M., Slaghek, T. M., Hypponen, T. K., Kruiskamp, P. H., Ogawa, T., Kamerling, J. P., and Vliegenthart, J. F. (1998) Carbohydr Res 309,161-174.
19. Heldermon, C., DeAngelis, P. L. and Weigel, P. H. (2001) J. Biol. Chem., 276, 2037-2046.
20. Hodson, N., Griffiths, G., Cook, N., Pourhossein, M., Gottfridson, E., Lind, T., Lidholt, K. and Roberts, I. S. (2000) J. Biol. Chem., 275. 27311-27315.
21. Jing, W., and DeAngelis, P. L. (2000) Glycobiology 10, 883-889.
22. Jing, W., and DeAngelis, P. L. (2003) Glycobiology 13, 661-671.
23. Jing, W., and DeAngelis, P. L. (2004) J Biol Chem 279, 4234542349.
24. Johnson, L. N., Cheetham, J., McLaughlin, P. J., Acharya, K. R., Barford, D., and Phillips, D. C. (1988) Curr Top Microbiol Immunol 139, 81-134.
25. Kitagawa. H., Uyama, T, and Sugahara, K. (2001) J. Biol. Chem. 276:38721-6.
26. Kumagai, I., Sunada, F., Takeda, S., and Miura, K. (1992) J Biol Chem 267, 46084612.
27. Kumari, K. and Weigel, P. H. (1997) J. Biol. Chem., 272, 32539-32546.
28. Lee, H. G., and Cowman, M. K. (1994) Anal Biochem 219, 278-287.
29. Leemhuis, H., Uitdehaag, J. C., Rozeboom, H. J., Dijkstra, B. W., and Dijkhuizen, L. (2002) J Biol Chem 277, 1113-1119.
30. Negishi, M., Dong, J., Darden, T. A., Pedersen, L. G., and Pedersen, L. C. (2003) Biochem Biophys Res Commun 303, 393-398.
31. Ninomiya T, Sugiura N, Tawada A, Sugimoto K, Watanabe H, Kimata K. (2002) J Biol Chem. 277:21567-75.
32. Pedersen, L. C., Tsuchida, K., Kitagawa, H., Sugahara, K., Darden, T. A., and Negishi, M. (2000) J Biol Chem 275, 34580-34585.
33. Pedersen, L. C., Dong, J., Taniguchi, F., Kitagawa, H., Krahn, J. M., Pedersen, L. G., Strynadka, N. C., and James, M. N. (1991) J Mol Biol 220, 401-424.
34. Petit, C., Rigg, G. P., Pazzani, C., Smith, A., Sieberth, V., Stevens, M., Boulnois, G., Jann, K. and Roberts, I. S. (1995) Mol. Microbiol., 17, 611-620.
35. Rimler, R. B. (1994) Vet. Rec., 134,191-192.
36. Stoolmiller, A. C. and Dorfman, A. (1969) J. Biol. Chem., 244, 236-346.
37. Sugahara, K., Schwartz, N. B. and Dorfman, A. (1979) J. Biol. Chem., 254, 6252-6261.
38. Sugahara, K., and Negishi, M. (2003) J Biol Chem 278, 14420-14428.
39. Tlapak-Simmons, V. L., Kempner, E. S., Baggenstoss, B. A. and Weigel, P. H. (1998) J. Biol. Chem., 273, 26100-26109.
40. Townsend, K. M., Boyce, J. D., Chung, J. Y., Frost, A. J., and Adler, B. (2001) J. Clin. Microbiol., 39, 924-929.
41. van Aalten, D. M. F., Komander, D., Synstad, B., Gaseidnes, S., Peter, M. G., Eijsink, V. G. H. (2001) Proc Natl Acad Sci USA 98, 8979-8984.
42. Vann, W. F., Schmidt, M. A., Jann, B. and Jann, K. (1981) Eur. J. Biochem., 116,359-364.
43. Weigel, P. H., Hascall, V. C. and Tammi, M. (1997) J. Biol. Chem., 272, 3997-4000.

What is claimed is:

1. A method for producing a glycosaminoglycan polymer derivative, comprising the steps of:
providing an enzymatically active glycosaminoglycan synthase enzyme from Pasteurella multocida;
providing a synthetic, artificial acceptor for the glycosaminoglycan synthase enzyme;
combining the synthetic, artificial acceptor with the glycosaminoglycan synthase enzyme within a reaction medium, wherein the reaction medium contains at least one sugar precursor selected from the group consisting of UDP-GlcA, UDP-GlcNAc, UDP-GalNAc; and
reacting the glycosaminoglycan synthase enzyme with the synthetic, artificial acceptor to produce an oligosaccharide or polysaccharide polymer derivative.

2. The method of claim 1, wherein the oligosaccharide or polysaccharide polymer derivative is selected from the group consisting of a hyaluronic acid (hyaluronan) polymer derivative, a chondroitin polymerderivative, a heparosan polymer derivative, and combinations thereof.

3. The method of claim 1, wherein the glycosaminoglycan synthase enzyme is selected from the group consisting of hyaluronan synthase, chondroitin synthase, heparosan synthase and combinations thereof.

4. The method of claim 1, wherein the synthetic, artificial acceptor comprises at least one monosaccharide attached to an organic hydrophobic molecule.

5. The method of claim 1, wherein the synthetic, artificial acceptor comprises two GlcA sugars attached to an aromatic nucleus.

6. The method of claim 1, wherein the synthetic, artificial acceptor is selected from the group consisting of fluorescein di-β-D-glucuronide (A-F-A), β-trifluoromethylumbelliferyl β-D-glucuronide (A-F₃MUM), and 4-methylumbelliferyl N-acetyl-β-D-glucosaminide (N-MUM).

7. The method of claim 6, wherein the synthetic, artificial acceptor is A-F-A.

8. A method for producing a hyaluronic acid (hyaluronan) polymer derivative, comprising the steps of:
providing an enzymatically active hyaluronan synthase enzyme from Pasteurella multocida;
providing a synthetic, artificial acceptor for the hyaluronan synthase enzyme;
combining the synthetic, artificial acceptor with the hyaluronan synthase enzyme within a reaction medium, wherein the reaction medium contains at least one sugar precursor selected from the group consisting of UDP-GlcA and UDP-GlcNAc; and
reacting the hyaluronan synthase enzyme with the synthetic, artificial acceptor to produce an hyaluronic acid (hyaluronan) polymer derivative.

9. The method of claim 8, wherein the synthetic, artificial acceptor comprises at least one monosaccharide attached to an organic hydrophobic molecule, wherein the monosaccharide is selected from the group consisting of GlcA, GlcNAc and GalNAc.

10. The method of claim 8, wherein the synthetic, artificial acceptor comprises two GlcA sugars attached to an aromatic nucleus.

11. The method of claim 8, wherein the synthetic, artificial acceptor is A-F-A.

12. A method for producing a chondroitin polymer derivative, comprising the steps of:
providing an enzymatically active chondroitin synthase enzyme from Pasteurella multocida;
providing a synthetic, artificial acceptor for the chondroitin synthase enzyme;
combining the synthetic, artificial acceptor with the chondroitin synthase enzyme within a reaction medium, wherein the reaction medium contains at least one sugar precursor selected from the group consisting of UDP-GlcA and UDP-GalNAc; and
reacting the chondroitin synthase enzyme with the synthetic, artificial acceptor to produce a chondroitin polymer derivative.

13. The method of claim 12, wherein the synthetic, artificial acceptor comprises at least one monosaccharide attached to an organic hydrophobic molecule, wherein the monosaccharide is selected from the group consisting of GlcA, GlcNAc and GalNAc.

14. The method of claim 12, wherein the synthetic, artificial acceptor comprises two GlcA sugars attached to an aromatic nucleus.

15. The method of claim 12, wherein the synthetic, artificial acceptor is A-F-A.

16. A method for producing a heparosan polymer derivative, comprising the steps of:
providing an enzymatically active heparosan synthase enzyme from Pasteurella multocida;
providing a synthetic, artificial acceptor for the heparosan synthase enzyme;
combining the synthetic, artificial acceptor with the heparosan synthase enzyme within a reaction medium, wherein the reaction medium contains at least one sugar precursor selected from the group consisting of UDP-GlcA and UDP-GlcNAc; and
reacting the heparosan synthase enzyme with the synthetic, artificial acceptor to produce a heparosan polymer derivative.

17. The method of claim 16, wherein the synthetic, artificial acceptor comprises at least one monosaccharide attached to an organic hydrophobic molecule, wherein the monosaccharide is selected from the group consisting of GlcA, GlcNAc and GalNAc.

18. The method of claim 16, wherein the synthetic, artificial acceptor comprises two GlcA sugars attached to an aromatic nucleus.

19. The method of claim 16, wherein the synthetic, artificial acceptor is A-F-A.
